(19) United States
(12) Patent Application Publication
(10) Pub. No.: US 2005/0009782 A1
(43) Pub. Date: Jan. 13, 2005

(54) Antiviral charged polymers that exhibit resistance to lysosomal degradation during kidney filtration and renal passage, compositions and methods of use thereof

(76) Inventor: Wayne D. Comper, North Ringwood (AU)

Correspondence Address: JONES DAY, 222 EAST 41ST ST, NEW YORK, NY, 10017, US

(21) Appl. No.: 10/888,594

(22) Filed: Jul. 9, 2004

Related U.S. Application Data
(60) Provisional application No. 60/485,670, filed on Jul. 9, 2003.

(51) Int. Cl. A61K 31/737

(52) U.S. Cl. 514/54

(57) ABSTRACT

The invention provides methods and compositions for treating, preventing or managing a viral infection in a subject comprising administering one or more sulfated polysaccharides, wherein the polysaccharides have a percent of sulfur with respect to the sugar residue effective to enable maximal interaction of constituent sulfate groups with the microbe which causes the infection and wherein the sulfated polysaccharide is not substantially endocytosed or degraded by cell receptor binding in the mammal and thereby retains antimicrobial activity in vivo. The invention also provides methods and compositions for treating, preventing or managing a non-viral. microbial infection in a subject comprising administering one or more substituted polysaccharides.

This application claims the benefit of U.S. Provisional Application No. 60/485,670, filed Jul. 9, 2003, which is incorporated herein by reference.

### 1. FIELD OF THE INVENTION

This invention relates to methods for treating or preventing viral infections in mammals using sulfated polysaccharides. In particular, this invention relates to methods of introducing a prophylactically or therapeutically effective amount of a charged and flexible sulfated polysaccharide into the blood stream, lymphatic system and/or extracellular spaces of a patient for the treatment, prevention or management of viral infections. More particularly, the invention provides methods of preventing, treating or managing a viral infection comprising administering a prophylactically or therapeutically effective amount of a charged polysaccharide into the blood stream, lymphatic system and/or extracellular spaces, wherein the percent of sulfur of the polysaccharide is effective to enable maximal interaction of the sulfate groups with the virus which causes the infection, and wherein the sulfated polysaccharide is not substantially endocytosed or degraded by cell receptor binding in the mammal, and thereby retains antiviral activity in vivo.

### 2. BACKGROUND OF THE INVENTION

Charged polysaccharides, particularly sulfated polysaccharides, have demonstrated potent antimicrobial activities in vitro (Baba et al., Antiviral Res 9:335-343, 1988; Ito et al., Antiviral Res. 7:1-367, 1987). For example, sulfated polysaccharides such as dextran sulfate, heparin, and pentosan polysulfate have been reported to be potent inhibitors of HIV, paramyxoviruses, cytomegaloviruses, influenza viruses, semlikiviruses (Lüscher-Mattli et al., Arch Virol 130:317-326, 1993) and herpes simplex viruses in vitro (Baba et al., Antimicrob. Agents Chemotherapy 32:1742-45, 1988; Pancheva, Antiviral Chem Chemotherapy 4:189-191, 1993). However, these known compounds have disappointingly poor activity in vivo.

Dextran sulfate and heparin were first reported to inhibit HIV replication in vitro by Ito et al., Antiviral Res. 7:36 1-367, 1987, Deringer et al. (U.S. Pat. No. 5,153,181) and Ueno and Kuno, Lancet 2:796-97, 1987. Later, several other sulfated polysaccharides were shown to inhibit HIV replication at concentrations believed to be below their respective cytotoxicity thresholds, e.g., pentosan sulfate (Baba et al., Antiviral Res 9: 335-343, 1988; Biesert et al., Aids 2: 449-57, 1988), fuciodan (Baba et al., Antiviral Res 9: 335-343, 1988), lambda-, kappa- and iota-carrageenan (Baba et al., Antiviral Res 9: 335-343, 1988), lentinan sulfate (Yoshida et al., Biochem. Pharmacol. 37:2887-91, 1988), mannan sulfate (Ito et al., Eur. J. Clin. Microbiol. Infect. Dis. 8: 191-193, 1989), dextrin sulfate (Ito et al. Antiviral Chem. Chemother., 2:41-44, 1991), sulfoeveman (Weiler et al., J Gen Virol 71:1957-1963, 1990), and sulfated cyclodextrins (Schols et al., J Acquired Immune Def. Syndr 4:677-85,1991.). However, these compounds have all proven ineffective in vivo, and at high concentrations cause thromobocytopenia, central nervous system side effects, hair loss, gastro-intestinal pain, anti-coagulation, and the like (Flexner et al., Antimicrob Agents Chemotherapy 35:2544-2550, 1991; Abrams et al., Annals of Internal Medicine (1989) 110:183-188; Hiebert et al., J. Lab & Clin. Med. 133:161-170 (1999)).

Conventional or commercial dextran sulfate has a percent of sulfation of about 17-22%. It is widely accepted that increasing sulfur content of dextran sulfate increases the anticoagulant activity of the material (Hirata et al., Biosci. Biotech. Biochem. 58: 406-407, 1994). Similarly, it is widely accepted that increasing the sulfur content of sulfated polysaccharides increases their in vitro antiviral activity. See, e.g., Witvrouw et al., General Pharmacology 29: 497-512, 1997; Nakashima et al., Jpn. J. Cancer Res. (Gann) 78: 1164-68, 1987; and Baba et al., J. AIDS 493-499, 1990. Again, these studies have demonstrated a marked increase in the in vitro activity of sulfated polysaccharides with the increase in sulfation, although the lack of in vivo efficacy remains. Indeed, lack of in vivo efficacy and the in vivo toxicity of compounds with a high degree of sulfation has been an unsolvable problem to date.

Although there have been a limited number of studies of sulfated polysaccharides with lower percents of sulfation for specific uses, these materials have not been characterized with respect to both their molecular weight and their percent of sulfation. Significantly, these materials have been reported to be less active against retroviruses than polysaccharides with 17-22% sulfation. Id. Further, poorly characterized (if characterized at all), low molecular weight preparations have been studied in animals for activity against herpes virus as in EP Application 0 066 379 A2 with limited success (See also, Pancheva SN. Antiviral Chem Chemotherapy 4:189-191, 1993.)

Considerable effort has been focused on improving the in vivo anti-viral activity of dextran sulfate by increasing its sulfation or modifying the use of conventional material. In one study, given the reported poor absorption of oral dextran sulfate, dextran sulfate was administered to a maximally tolerated dose by continuous infusion to subjects with symptomatic HIV infection for up to 14 days (Flexner et al., Antimicrob Agents Chemotherapy 35:2544-2550, 1991). Continuous intravenous infusion of dextran sulfate was found to be toxic. The authors concluded that as a result of its toxicity and lack of any demonstration of beneficial effect in vivo, dextran sulfate is unlikely to have a beneficial effect in the treatment of HIV. Id. Indeed, the authors cautioned: “further clinical development of parenteral dextran sulfate as therapy for symptomatic HIV infection is not warranted and could prove to be hazardous. On the basis of the results of this study, caution is advised in the clinical evaluation of other polysulfated polyanions.” (Id. at 2549).

In sum, although commercial dextran sulfate has been previously used in Japan for anticoagulation and hyperlipidemia, it has demonstrated poor activity against HIV in vivo or, dextran sulfate has been reported to have significant toxicity in mammals and HIV patients (Mathis et al., Antimicrobial Agents & Chemotherapy 2147-2150, 1991; Flexner et al., Id. 2544-2550; Abrams et al., Annals of Internal Medicine 110: 183-188 (1989); Hiebert et al., J. Lab & Clin. Med. 133:161-170 (1999)). Thus, there remains a need for a method for the in vivo activation of dextran sulfate against viral and other infections. There also remains a need for a sulfated polysaccharide that is effective in vivo for the treatment or prevention of viral infections.

### 3. SUMMARY OF THE INVENTION

Applicant has previously discovered that synthetic sulfated polysaccharides having a percent of sulfur between 6 and 13% are effective in vivo against microbial infections, particularly viruses (See, e.g., Applicant's copending U.S. application Ser. No. 10/321,756, filed Dec. 17, 2003). This discovery was made despite the accepted belief that dextran sulfate was either ineffective in vivo or toxic in vivo or both. The present application further contradicts the reports that naturally occurring sulfated polysaccharides lack in vivo efficacy against microbial infection in therapeutically or prohylactically effective amounts without excessive toxicity. Thus, this invention encompasses novel methods for the treatment or prevention of microbial infections (e.g., viral, bacteria, fungal, and parasite infections), and novel pharmaceutical compositions which utilize sulfated polysaccharides, including naturally occurring, non-synthetic and commercially available polysaccharides, particularly dextran sulfates effective against viral infection, particularly respiratory infection or viral infection leading to respiratory disease. The invention encompasses in a preferred embodiment the use of sulfated polysaccharides, having a percent of sulfur with respect to the simple sugar residue of greater than 2% and less than 25%, more preferably greater than 2% and less than 6% or greater than 13% and less than 25%, within the methods and compositions of the invention. The sulfated polysaccharides are preferably sulfated dextrans having an α-1,6-glycosidic linkage.

The invention further encompasses the use of sulfated polysaccharides having a molecular weight between 1000 and 1,000,000, preferably above 5,000, more preferably above 25,000, most preferably above 40,000 within the methods and compositions. Ranges of 5,000 to 1,000,000; 25,000 to 500,000, and 40,000 to 300,000 are also encompassed by the invention for oral or parenteral use. However, for topical administration, the sulfated polysaccharide may have a molecular weight of 5,000 to 1,000,000 but preferably higher than 500,000. In a specific embodiment, the composition has only about 10% variability in the molecular weight and preferably about 5% variation.

In addition to dextran sulfates, the sulfated polysaccharide can be cellulose sulfate, dextrin sulfate, cyclodextrin, or one of the other materials found in Table 1 below. Preferably the percent of sulfur of such sulfated polysaccharide is within the range of 2% to 25%, preferably greater than 2% and less than 6% or greater than 13% and less than 25%, more preferably greater than 14% and less than 22%, most preferably greater than 15% and less than 18%. Moreover, substituted polysaccharides such as co-charged anionic polysaccharides (such as, but not limited to, carboxymethyl substituted) or periodated treated sulfated polysaccharides, particularly substituted dextran sulfates such as carboxymethyl substituted dextran sulfate or periodate treated dextran sulfates can be used. In one embodiment, the sulfated polysaccharide is homogenous with respect to molecular weight, percent of sulfation or both.

The invention encompasses a method for treating, preventing or managing a viral infection comprising administering a prophylactically or therapeutically effective amount of a sulfated polysaccharide or a substituted polysaccharide (particularly a co-charged anionic polysaccharide) or a salt thereof to a subject (preferably, a human) in need thereof, wherein the sulfated or substituted polysaccharide has a percent sulfur above 2% but less than 6% or above 13% but less than 25% with respect to the simple sugar residue. Preferably, the percent of sulfur of the polysaccharide is effective to enable maximal interaction of constituent sulfate groups with the virus which causes the viral infection, and wherein the sulfated polysaccharide is not substantially endocytosed or degraded by cell receptor binding in the subject, and thereby retains antiviral activity in vivo. In order words, the preferred sulfated polysaccharides of the invention are active and relatively non-toxic in vivo. In a specific embodiment, the sulfated polysaccharide or substituted polysaccharide is administered into the blood stream, lymphatic system and/or extracellular spaces of the subject.

Non-limiting examples of the viral infection prevented, managed or treated in accordance with the methods of the invention include, DNA viral infections and RNA viral infections. In a specific embodiment, the virus is an enveloped virus (e.g., a DNA or RNA enveloped virus). In another embodiment, the virus is a double-stranded DNA virus, a DNA reverse transcripting virus, an RNA reverse transcripting virus, a double-stranded RNA virus, a negative-sense single stranded RNA virus, or a positive-sense single-stranded RNA virus.

In a specific embodiment of the invention, the viral infection prevented, treated or managed is not a herpes virus infection, or more specifically a HSV-1 and/or HSV-2 infection. In another alternative embodiment, the viral infection prevented, treated or managed is not a retrovirus infection, or more specifically, a HIV-1, HIV-2 and/or HTLV infection. Further, in another embodiment, the viral infection prevented, treated or managed is not a hepatitis B virus, HCMV, MCMV, VZV, EBV, Measles virus, Punto Toro a, VEE, West Nile Virus, Vaccinia, Cow pox, Adenovirus Type 1, HPIV, Human metapneumoviurs, Haemorrhagic septicaemia virus, Parainfluenza type 3, Pichinde and/or rhinovirus infection.

The invention encompasses a method of treating, preventing or managing a bacterial infection (including, but not limited to gram positive bacterial infection and gram negative bacterial infection), fungal infection or parasitic infection in a subject (preferably, a human) comprising administering to a subject in need thereof a therapeutically effective amount of a naturally occurring, synthetic, or non-synthetic sulfated polysaccharide or a salt thereof, or a substituted polysaccharide (e.g., a co-charged anionic polysaccharide) or a salt thereof, wherein the sulfated or substituted polysaccharide or a salt thereof has a percent of sulfur above 2% but less than 6% or above 13% but less than 25% with respect to the simple sugar residue.

In one embodiment, the sulfated polysaccharides are used in combination with another therapy to prevent, treat or manage a microbial infection (e.g, a viral, bacterial, fungal or parasitic infection). In a specific embodiment, the sulfated polysaccharides are used in combination with a molecule which targets the area of the infection or otherwise reduces the in vivo toxicity of the sulfate polysaccharide.

The invention also encompasses pharmaceutical compositions suitable for parenteral administration to a patient comprising a therapeutically or prophylactically acceptable amount of a sulfated polysaccharide of the invention in a sterile form; pharmaceutical compositions suitable for oral administration to a patient comprising a therapeutically or prophylactically acceptable amount of a sulfated polysaccharide of the invention; and pharmaceutical compositions suitable for topical administration to a patient comprising a therapeutically or prophylactically acceptable amount of a sulfated polysaccharide of the invention, preferably having a molecular weight greater than 500,000. Depending on the specific tissue to undergo therapy, additional components, such as penetration enhancers, may be used prior to, in conjunction with, or subsequent to the administration with active ingredients of the invention. In a preferred embodiment, each of these compositions is in single unit dosage form and comprising an amount of active ingredient sufficient to treat, prevent or manage human infection by virus, preferably a non-retrovirus.

The invention also encompasses the use of the sulfated polysaccharides of the invention as disinfectants that can be used to disinfect inanimate objects particularly in hospitals, laboratories, lavatories, auditoriums, stadiums, convention centers, restaurants, fitness centers, subway terminals, bus terminals, airports, post offices, offices, sewage treatment facilities, sewers, water treatment facilities, pumping stations, automobiles, airplanes, trains, homes, lockers, and furniture to prevent the spread of the virus. The invention also encompasses disinfectant compositions such as solutions, gels, powders, concentrates, lotions, creams, sprays, aerosols, soaps, or foams comprising one or more of the sulfated polysaccharides described herein.

3.1 Definitions

As used herein, the term “patient” or “subject” means an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an infant, child, adolescent, adult or geriatric patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

As used herein, the term “geriatric patient” refers to a human 65 years old or older, preferably 70 years old or older.

As used herein, the term “adult patient” refers to a human between 18 years of age and 65 years of age.

As used herein, the term “adolescent patient” refers to a human between 18 years of age and 13 years of age.

As used herein, the term “child patient” refers to a human between 24 months of age and 13 years of age.

As used herein, the term “infant patient” refers to a human less than 24 months, preferably less than 16 months, less than 6 months, less than 3 months, less than 2 months, or less than 1 month of age.

As used herein, the phrase “infection” includes the invasion by and/or multiplication of a microbe in a cell or body tissue, and the pathological state resulting from the invasion by and multiplication of a microbe (e.g., virus, bacterium, fungus, or parasite). The invasion by and multiplication steps of a virus' life cycle include, but are not limited to, the following steps: the docking of the virus particle to a cell, the introduction of viral genetic information into a cell, the expression of viral proteins, the production of new virus particles and the release of virus particles from a cell.

As used herein, the term “effective amount” refers to the amount of a therapy (e.g., a sulfated polysaccharide of the invention or another prophylactic or therapeutic agent) which is sufficient to reduce or ameliorate the severity and/or duration of a microbial infection or one or more symptoms thereof, prevent the advancement of a microbial infection, prevent the recurrence, development, or onset of one or more symptoms associated with a microbial infection, prevent or reduce the replication or multiplication of a microbe (e.g, a virus), prevent or reduce the production and/or release of a viral particle, or enhance or improve the prophylactic or therapeutic effect(s) of another therapy (e.g., prophylactic or therapeutic agent). In a specific embodiment, an effective amount of a therapy reduces one or more of the following steps of a the life cycle of a virus: the docking of the virus particle to a cell, the introduction of viral genetic information into a cell, the expression of viral proteins, the production of new virus particles and the release of virus particles from a cell by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another specific embodiment, an effective amount of a therapy reduces the replication, multiplication or spread of a microbe by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

As used herein, a “therapeutically effective amount” refers to an amount of a therapy (e.g., a sulfated polysaccharide of the invention) sufficient to provide a benefit in the treatment or management of a microbial infection, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

As used herein, a “prophylactically effective amount” refers to an amount of a therapy (e.g., a sulfated polysaccharide of the invention or other therapeutic or prophylactic agent) sufficient to result in the prevention of infection, recurrence or spread of a microbial infection. A prophylactically effective amount may refer to an amount sufficient to prevent initial infection or the recurrence or spread of the infection. Used in connection with an amount of a compound of the invention, the term preferably encompasses a non-toxic amount that improves overall prophylaxis or enhances the prophylactic efficacy of or synergies with another prophylactic or therapeutic agent.

As used herein, the term “in combination” refers to the use of more than one therapy (e.g., more than one prophylactic agent and/or therapeutic agent). The use of the term “in combination” does not restrict the order in which therapies (e.g., prophylactic and/or therapeutic agents) are administered to a subject with a microbial infection. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject with a microbial infection.

As used herein, the terms “manage,” “managing,” and “management” refer to the beneficial effects that a subject derives from a therapy (e.g., a prophylactic or therapeutic agent), which does not result in a cure of a microbial infection. In certain embodiments, a subject is administered one or more therapies (e.g., one or more prophylactic or therapeutic agents) to “manage” a disease so as to prevent the progression or worsening of a microbial infection.

As used herein, the terms “non-responsive” and refractory” describe patients treated with a currently available therapy (e.g., prophylactic or therapeutic agent) for a microbial infection which is not clinically adequate to relieve one or more symptoms associated with the infection. Typically, such patients suffer from severe, persistently active infection and require additional therapy to ameliorate the symptoms associated with the infection.

As used herein, the terms “prevent”, “preventing” and “prevention” refer to the prevention of the recurrence, onset or development of one or more symptoms of a microbial infection in a subject resulting from the administration of a therapy (e.g., a prophylactic or therapeutic agent), or the administration of a combination of therapies (e.g., a combination of prophylactic or therapeutic agents).

As used herein, the terms “prophylactic agent” and “prophylactic agents” refer to any agent(s) which can be used in the prevention of a microbial infection. In certain embodiments, the term “prophylactic agent” refers to a sulfated polysaccharide. In other embodiments, the term “prophylactic agent” refers to a substituted polysaccharide (e.g., a co-charged anionic polysaccharide). In certain other embodiments, the term “prophylactic agent” does not refer a sulfated polysaccharide and/or a substituted polysaccharide.

As used herein, the term “prophylactically effective amount” refers to the amount of a therapy (e.g., prophylactic agent) which is sufficient to result in the prevention of the development, recurrence, or onset of a microbial infection or one or more symptoms thereof, or to enhance or improve the prophylactic effect(s) of another therapy (e.g., a prophylactic agent). In a specific embodiment, a prophylactically effective amount of a prophylactic agent reduces one or more of the following steps of the life cycle of a virus: the docking of the virus particle to a cell, the introduction of viral genetic information into a cell, the expression of viral proteins, the production of new virus particles and the release of virus particles from a cell by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, a prophylactically effective amount of a therapy reduces the replication, multiplication or spread of a microbe by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

As used herein, the term “purified” refers to a prophylactic or therapeutic agent (e.g., a sulfated polysaccharide or a substituted polysaccharide such as a co-charged anionic polysaccharide) substantially free of a different prophylactic or therapeutic agent. Preferably, a prophylactic or therapeutic agent is at least 60%, 65%, 70%, 75%, 80%, 85%, 90%, 95%, or 99% free of a second, different prophylactic or therapeutic agent.

As used herein, the phrase “side effects” encompasses unwanted and adverse effects of a therapy. Adverse effects are always unwanted, but unwanted effects are not necessarily adverse. An adverse effect from a therapy might be harmful or uncomfortable or risky.

As used herein, the term “synergistic” refers to a combination of a sulfated polysaccharide or a substituted polysaccharide, such as a co-charged anionic polysaccharide, and another therapy (e.g., a prophylactic or therapeutic agent), which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a microbial infection. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention, management or treatment of a microbial infection. In addition, a synergistic effect can result in improved efficacy of therapies (e.g., prophylactic or therapeutic agents) in the prevention, management or treatment of a microbial infection. Finally, synergistic effect of a combination of therapies (e.g., prophylactic or therapeutic agents) may avoid or reduce adverse or unwanted side effects associated with the use of any single therapy.

As used herein, the terms “therapeutic agent” and “therapeutic agents” refer to any agent(s) which can be used in the prevention, treatment, management or amelioration of a microbial infection or one or more symptoms thereof. In certain embodiments, the term “therapeutic agent” refers to a sulfated polysaccharide. In other embodiments, the term “therapeutic agent” refers to a substituted polysaccharide (e.g., a co-charged anionic polysaccharide). In other embodiments, the term “therapeutic agent” does not refer to a sulfated polysaccharide and/or a substituted polysaccharide.

As used herein, the term “therapeutically effective amount” refers to that amount of the therapeutic agent which is sufficient to reduce the severity of a microbial infection, reduce the duration of a microbial infection, ameliorate one or more symptoms of a microbial infection, prevent the advancement of a microbial infection, cause regression of a microbial infection, or to enhance or improve the therapeutic effect(s) of another therapy. With respect to the treatment of a viral infection, a therapeutically effective amount refers to the amount of a therapeutic agent sufficient to reduce or inhibit the replication of a virus, inhibit or reduce the infection of a cell with the virus, inhibit or reduce the production of the viral particles, inhibit or reduce the release of viral particles, inhibit or reduce the spread of the virus to other tissues or subjects, or ameliorate one or more symptoms associated with the infection. In a specific embodiment, a therapeutically effective amount of a therapeutic agent reduces one or more of the following steps of a the life cycle of a virus: the docking of the virus particle to a cell, the introduction of viral genetic information into a cell, the expression of viral proteins, the production of new virus particles and the release of microbe particles from a cell by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, a therapeutically effective amount of a therapeutic agent reduces the replication, multiplication or spread of a microbe by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

As used herein, the terms “therapies” and “therapy” refer to any protocol(s), method(s), and/or agent(s) that can be used in the prevention, treatment, management, or amelioration of a microbial infection or one or more symptoms thereof. In certain embodiments, the terms “therapy” and “therapy” refer to anti-viral therapy, antibiotic therapy, biological therapy, supportive therapy, and/or other therapies useful in treatment, management, prevention, or amelioration of a microbial infection or one or more symptoms thereof known to skilled medical personnel.

As used herein, the terms “treat”, “treatment” and “treating” in the context of treating a microbial infection refer to the reduction or amelioration of the progression, severity, and/or duration of a microbial infection or the amelioration of one or more symptoms thereof resulting from the administration of one or more therapies (including, but not limited to, the administration of one or more prophylactic or therapeutic agents). In specific embodiments, such terms refer to the reduction or inhibition of the replication of a microbe, the inhibition or reduction in the spread of a microbe to other tissues or subjects, the inhibition or reduction of infection of a cell with a microbe, or the amelioration of one or more symptoms associated with a microbial infection.

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of the present invention include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

As used herein and unless otherwise indicated, the term “optically pure” or “stereomerically pure” means a composition that comprises one stereoisomer of a compound and is substantially free of other stereoisomers of that compound. For example, a stereomerically pure a compound having one chiral center will be substantially free of the opposite enantiomer of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. Since the compounds of the invention are primarily polysaccharides made of saccharides which can exist in either the D or L forms, the invention encompasses either or both D and L sugars. As such, for example, a stereomerically pure D sugar will be substantially free of the L form. In an alternative embodiment, the use of L forms of sulfated dextrans permits the use of a broader controlled range of sulfation from above 2% to about 30%. Thus, the methods and compositions disclosed herein include in an alternative embodiment the use of such levorotatory sugars or polymers made therefrom.

As used herein, the term “sulfated polysaccharide” means a naturally occurring, non-synthetic, or synthetic sulfated material having more than five units of simple sugar. Preferably, the sulfated polysaccharide is an alpha(1,6) linked polysaccharide. The sulfated polysaccharides of the invention also preferably have a percent of sulfur that is sufficient for both in vitro and in vivo activity without significant toxicity.

As used herein, the term “dextran” means a polysaccharide containing a backbone of D-glucose units linked predominantly α-D(1,6), composed exclusively of α-D-glucopyranosyl units differing only in degree of branching and chain length.

As used herein, the term “dextran sulfate sodium” or “dextran sulfate”, “conventional dextran sulfate”, or “commercial dextran sulfate” unless otherwise qualified means a α-1,6-polyglucose containing approximately 17% sulfur with up to three sulfate groups per glucose molecule of varying molecular weight ranges, e.g., 4,000-500,000 Da.

As used herein, the terms “percent sulfation”, “percent of sulfation”, “percent of sulfate substitution” or “sulfation” means the percent of sulfur by molecular weight with respect to each simple sugar residue within the polysaccharide in question, optionally including a counterion, e.g., molecular weight of sulfation in the composition/total weight. In a preferred embodiment, the percent of sulfur is calculated as the percent of sulfur by molecular weight with respect to the sulfated sugar residue within the polysaccharide in question with sodium as the counterion. The percent of sulfation can be determined by elemental analysis of material which has been dialyzed to remove free sulfur, preferably of moisture/volatile free material dried in vacuo at 60° C. to a constant weight. Other methods of determining percent of sulfation are via moisture content analysis and titration. Sulfation is to be distinguished from “degree of substitution” or “equivalents” which is a measure of the number of sulfate groups per sugar moiety. However, it will be recognized by one of skill in the art that percent sulfation can be converted to a degree of substitution or equivalents and vice versa.

As used herein, the term “co-charged dextran polyanions” is dextran substituted to varying degrees with any combination of carboxymethyl groups, sulfate groups and sulfonate groups.

As used herein, the term “periodate treated anionic polysaccharides” means any anionic polysaccharide that has been treated with periodate to open the sugar ring without depolymerization or to otherwise increase the flexibility of the polysaccharide in order to increase interaction with the virus.

### 4. BRIEF DESCRIPTION OF THE DRAWINGS

FIG. 1 is a schematic flowchart describing the preparation of sulfated dextrans of a specific percent of sulfation and molecular weights.

### 5. DETAILED DESCRIPTION OF THE INVENTION

Contrary to the teachings in the literature, the present invention encompasses the in vivo use of sulfated polysaccharides, including those with a range of sulfur content below or above 6% to 13% to treat microbial infections. In one embodiment of the invention, the invention encompasses sulfated polysaccharides such as conventional dextran sulfate or variants thereof (e.g., dextran sulfate with a percent of sulfur that differs from the conventional material) that can be used to treat, prevent or manage microbial infection. In one embodiment, the sulfated polysaccharide has a percent of sulfation greater than 2% but below 25% range, preferably greater than 2% and less than 6% or greater than 13% and less than 25%, more preferably greater than 14% and less than 22%, most preferably greater than 15% and less than 18%. The most preferred compositions or methods of the invention utilize sulfated α-1,6-linked polysaccharides or sulfated dextrans having the desired percent of sulfation and/or molecular weight which are flexible and thus useful against a wide variety of viruses. In a most preferred embodiment, the range of percent sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo.

Thus, the present invention encompasses methods for treating, preventing or managing a microbial infection (e.g., a viral infection) in vivo, with a sulfated polysaccharide or a pharmaceutically acceptable salt, hydrate, or stereoisomer thereof, having flexibility in its structure, a controlled degree of sulfation, and optionally homogeneity as to its molecular weight, and low degree of sulfation as compared to conventional dextran sulfate.

The present invention provides methods for the treatment, prevention, or management of a microbial infection (e.g., a viral infection) comprising administering to a patient in need thereof a therapeutically or prophylactically effective amount of a sulfated polysaccharide or pharmaceutically acceptable salts, hydrates, or stereoisomers thereof having from greater than 2% but less than 25% sulfation.

Without being limited by any particular theory, the Applicant believes that there is a range of charge density for sulfated polysaccharides within which they exhibit anti-microbial activity in vitro and retain their anti-microbial activity (e.g., anti-viral activity) in vivo. In a preferred embodiment of the invention, the sulfated polysaccharides of the invention have a percent of sulfation of greater than 2% but less than 6% or greater than 13% but less than 25%, preferably greater than about 14% but less than 22%, specifically 2.5%, 3%, 3.5%, 4%, 4.5%, 5%, 5.5%, 13.5%, 14%, 14.5%, 15%, 15.5%, 16%, 16.5%, 16.8%, 17%, 17.5%, 18%, 18.5%, 19%, 19.5%, 20%, 20.5%, 21%, 21.5%, 22%, 22.5%, 23%, 23.5%, 24% or 24.5% within ±1%.

A preferred sulfated polysaccharide used in the methods of the invention is sulfated dextran, or an α-1,6-linked polysaccharide, which has been modified to have the appropriate percent of sulfation. The sulfated dextran of the invention contain less than 25% sulfur, and may contain less than 17% sulfur, less than 16% sulfur, less than about 15% sulfur, less than 14% sulfur, but more than 13% sulfur. The sulfated dextran of the invention may also contain less than 6%, and may contain less than 5%, less than 3%, less than about 3%, but more than 2% sulfur. In a preferred embodiment, the sulfated dextran variant has a sulfation of less than 17% and greater than 13%.

The invention further encompasses the use of sulfated polysaccharides having a molecular weight between 1000 and 1,000,000, preferably above 5,000; more preferably above 25,000; more preferably above 40,000; and specifically 7,500, 10,000, 12,500, 15,000, 17,500, 20,000, 22,500, 25,000 27,500, 30,000, 32,500, 35,000, 37,500, 40,000, 42,500, 45,000, 47,500; 50,000, 52,500, 55,000, 57,500 or 60,000 within the methods and compositions. Ranges of 5,000 to 1,000,000; 25,000 to 500,000; and 40,000 to 300,000 are also encompassed by the invention for oral or parenteral use. However, for topical administration, the sulfated polysaccharide may have a molecular weight of 5,000 to 1,000,000 but preferably higher than 500,000. In a specific embodiment, the composition has only about 10% variability in the molecular weight and preferably about 5% variation.

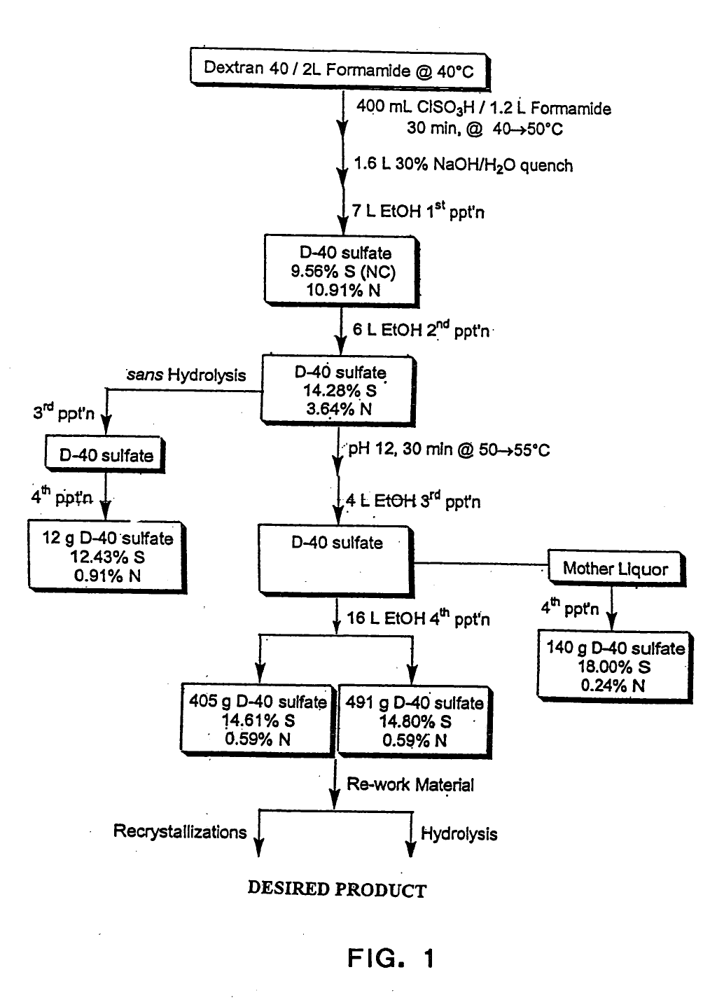

The sulfated polysaccharides of the invention can be naturally occurring, non-synthetic or synthetic. The synthetic sulfated polysaccharides, particularly the sulfated dextrans, can be prepared using known synthetic techniques and reagents. Several methods which are known in the art may be modified so that the proper degree of sulfation is achieved. These methods include those described in FIG. 1. However, as mentioned above, one may control the molecular weight as well as the degree of sulfation. Applicant has synthesized sulfated dextran with controlled sulfur contents and controlled degrees of sulfate substitution so that they are not taken up by cell receptors for highly charged polysaccharides. These polysaccharides exhibit essentially the same antiviral activity in vivo as they do in vitro and have enhanced stability and longevity in vivo, as they are not readily taken up by cells they are also less toxic. Sulfated dextran, with controlled sulfur content is particularly well suited as a viral cell attachment inhibitor because of its unique structure—essentially linear chain composed of an α-1,6-glycosidic linkage which makes it a more flexible polysaccharide—that enables maximal interaction of its constituent sulfate groups with positive charges on proteins of the virus but does not bind significantly to plasma proteins including albumin.

The invention also encompasses the use of homogeneous sulfated polysaccharides. That is to say the sulfated polysaccharides administered in accordance with the methods described herein or utilized in the pharmaceutical compositions and dosage forms exhibit substantially the same percent of sulfation or molecular weight or both.

The invention provides a method of treating, managing or preventing a viral infection in a mammal comprising administering to a subject (preferably, a human) in need thereof an effective amount of a composition comprising a sulfated polysaccharide having a percent of sulfate substitution per glucose residue in the polysaccharide ranging from greater than 2% to less than 25%, wherein the range of percent sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo. Preferably, the sulfated polysaccharide is sulfated dextran.

The invention also encompasses the treatment, prevention or management of anti-inflammatory diseases or disorders, interstitial cystisis and anti-arthritic diseases. The invention also encompasses the use of the sulfated polysaccharides of the invention as anti-albuminuric agents (albuminuria that occurs in kidney disease).

The invention provides a method of treating, managing or preventing a microbial infection (e.g., a viral infection) in a subject (preferably, a human) which comprises administering to a subject in need thereof an effective amount of a levorotatory sulfated polysaccharide having a percent of sulfation from about 2% to about 25%; preferably from about 2% to about 6%, or from about 13% to about 20%; and more preferably from about 14% to about 17%.

In a specific embodiment, the invention encompasses a method of treating, managing or preventing a microbial infection (e.g., a viral infection) in a subject (e.g, preferably, a human) which comprises administering to a subject in need thereof an effective amount of a periodate-treated anionic polysaccharide. Preferably, the periodate treated anionic polysaccharide is a periodate treated sulfated dextran.

In one embodiment, the invention provides a method of treating, managing or preventing a microbial infection comprising administering to a subject (preferably, a human) an effective amount of a substituted polysaccharide such as a co-charged anionic polysaccharide). In another embodiment of the invention, the invention provides a method of treating, managing, or preventing a viral infection in a subject (preferably, a human) which comprises administering to a subject in need of thereof an effective amount of a co-charged anionic polysaccharide which has a percent of sulfation which enables maximal interaction with the virus and which is not substantially endocytosed or degraded by cell receptor binding in the mammal thereby retaining antiviral in vivo. In a preferred embodiment, the co-charged anionic polysaccharide is co-charged with carboxymethyl groups, sulfonate groups, sulfate groups or mixtures thereof; more preferably the co-charged anionic polysaccharide is co-charged with carboxymethyl groups. In a specific embodiment, the co-charged anionic polysaccharide is carboxymethyl dextran sulfate or carboxymethyl cellulose.

In another embodiment of the invention, and depending on the specific tissue to be treated, additional components; including, but not limited to penetration enhancers, molecules which targets the area of the infection and molecules which reduce the in vivo toxicity of the sulfate polysaccharide; may be used prior to, in conjunction with, or subsequent to treatment with one or more sulfated polysaccharides and/or substituted polysaccharides of the invention.

5.1 Methods of Activating Sulfated Polysaccharides For In Vivo Use

The invention encompasses a method of increasing or decreasing sulfation of naturally occurring sulfated polysaccharides for administration in vivo comprising providing the sulfated polysaccharide with a sulfation sufficient to eliminate or reduce binding of the sulfated polysaccharide by high charge density polyanion cell receptors and to provide anti-viral activity to the sulfated polysaccharide. The sulfation range can be reached by preparation of compositions with the desired percent of sulfation. Alternatively, naturally occurring material can be modified or controlled chemically or enzymatically to the degree of sulfation range wherein the sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo.

Listed in Table 1 below are examples of sulfated polysaccharides (not including dextran sulfate) whose anti-viral activity has been demonstrated in vitro, but which previously have not been shown to have anti-viral activity in vivo at a dosage below the cytotoxicity level of these compounds.

TABLE 1
Sulfated Polysaccharides having anti-viral
or anti-bacterial activity in vitro

| Sulfated polysaccharides | In vitro activity |
|---|---|
| (14)-2-deoxy-2-sulfamido-3-O-sulfo-(14)-beta-D- | HIV |
| glycopyranan (derivative of chitosan) | |
| 2-acetamido-2-deoxy-3-O-sulfo(14)-beta-D- | HIV |
| glycopyranan (derivative of chitosan) | |
| Achranthese bidentata polysaccharide sulfate | HSV-1 |
| Aurintricarboxylic acid | HIV |
| Calcium spirulan | HIV, CMV, HSV-1, |
| | measles, mumps, |
| | influenza type A |
| Carboxymethylchitin | Friend murine |
| | leukemia virus, |
| | HSV |
| Chemically degraded heparin (Org 31733) | HIV, HHV-7 |
| Chondroitin polysulfate | HIV |
| Copolymer of sulphonic acid and biphenyl | HIV |
| disulphonic acid urea (MDL 10128) | |
| Curdlan sulfate | HIV, CMV |
| Cyanovirin-N (from cyanobacterium) | HIV |
| Fucoidin | HIV, Chlamydia, |
| | ASFV |
| Galactan sulfate | HIV, HSV-1 |
| Glucosamine-6-sulfate (monosaccharide) | HIV |
| Glycyrrhizin sulfate | HIV |
| Heparin | HIV, HHV-7, |
| | ASFV, Denge |
| | virus, MLV |
| Inositol hexasulfate | HIV |
| Lentinan sulfate | HIV |
| Mannan sulfate | HIV |
| N-acylated heparin conjugates | HIV |
| N-carboxymethylchitosan-N,O-sulfate | HIV, RLV |
| Oligonucleotide-poly(L-lysine)-heparin complexes | HIV |
| Pentosan polysulfate (xylanopolyhydrogen sulfate) | HIV, Chlamydia, |
| | ASFV |
| Peptidoglycan DS-4152 | HIV |
| Periodate degraded heparin | HIV |
| Phosphorothioate oligodeoxynucleotides | HIV |
| Polyacetal polysulfate | HIV |
| Polyinosinic-polycytidylic acid | HIV |
| Polysaccharides from Indocalamus tesselatus | HIV |
| (bamboo leaves) | |
| Prunellin | HIV |
| Rhamnan sulfate | HIV, HSV-1, CMV |
| Ribofuranan sulfate | HIV |
| Sodium lauryl sulfate | HIV, HSV |
| Sulfate dodecyl laminarapentaoside (alkyl | HIV |
| oligosaccharide) | |
| Sulfated bacterial glycosaminooglycan | HIV |
| Sulfated dodecyl laminari-oligomer (alkyl | HIV |
| oligosaccharide) | |
| Sulfated gangliosides | HIV |
| Sulfated laminara-oligosaccharide glycosides | HIV |
| synthesized from laminara-tetraose, laminara- | |
| pentaose, laminara-hexaose | |
| Sulfated N-deacetylatedchitin | Friend murine |
| | leukemia virus, |
| | HSV |
| Sulfated octadecyl maltohexaoside (alkyl | HIV |
| oligosaccharide) | |
| Sulfated octadecyl ribofurnans | HIV |
| Sulfated oligoxylan (heparin mimetic) | HIV |
| Sulfated xylogalactans | HIV-1 |
| Sulfatide (3′ sulfogalactosylceramide) | HIV |
| Sulfoevernan | HIV |
| Xylomannan sulfate | HIV, HSV-1, |
| | HSV-2 |

ASFV: African Swine Fever Virus;
HHV-7: Human Herpes Virus;
HSV: herpes simplex virus;
CMV: cytomegalovirus

Each of sulfated polysaccharides listed above, as well as any other sulfated polysaccharide that has anti-microbial (e.g., anti-viral activity) in vitro, may be modified to bring their degree of sulfation or ionic charge to a level suitable for their use in the methods or compositions of the invention.

The invention provides a method of treating, managing or preventing a microbial infection (e.g., a viral infection) in a subject (preferably, a human) which comprises administering to a subject in need thereof one or more compounds chosen from the group consisting of cellulose sulfate; (14)-2-deoxy-2-sulfamido-3-O-sulfo-(14)-beta-D-glycopyranan (derivative of chitosan); 2-acetamido-2-deoxy-3-O-sulfo(14)-beta-D-glycopyranan (derivative of chitosan); Achranthese bidentata polysaccharide sulfate; Aurintricarboxylic acid; Calcium spirulan; Carboxymethylchitin; Chemically degraded heparin (Org 31733); Chondroitin polysulfate; Copolymer of sulphonic acid and biphenyl disulphonic acid urea (MDL 10128); Curdlan sulfate; Cyanovirin-N (from cyanobacterium); Fucoidin; Galactan sulfate; Glucosamine-6-sulfate (monosaccharide); Glycyrrhizin sulfate; Heparin; Inositol hexasulfate; Lentinan sulfate; Mannan sulfate; N-acylated heparin conjugates; N-carboxymethylchitosan-N,O-sulfate; Oligonucleotide-poly(L-lysine)-heparin complexes; Pentosan polysulfate (xylanopolyhydrogen sulfate); Peptidoglycan DS-4152; Periodate degraded heparin; Phosphorothioate oligodeoxynucleotides; Polyacetal polysulfate; Polyinosinic-polycytidylic acid; Polysaccharides from Indocalamus tesselatus (bamboo leaves); Prunellin; Rhamnan sulfate; Ribofuranan sulfate; Sodium lauryl sulfate; Sulfate dodecyl laminarapentaoside (alkyl oligosaccharide); Sulfated bacterial glycosaminooglycan; Sulfated dodecyl laminari-oligomer (alkyl oligosaccharide); Sulfated gangliosides; Sulfated laminara-oligosaccharide glycosides synthesized from laminara-tetraose, laminara-pentaose, laminara-hexaose; Sulfated N-deacetylatedchitin; Sulfated octadecyl maltohexaoside (alkyl oligosaccharide); Sulfated octadecyl ribofurnans; Sulfated oligoxylan (heparin mimetic); Sulfated xylogalactans; Sulfatide (3′ sulfogalactosylceramide); Sulfoeveman; and Xylomannan sulfate. In a specific embodiment, with respect to a viral infection, the percent of sulfation of such a compound has been modified or controlled to enable maximal interaction of constituent sulfate groups with the virus causing the infection, and the compound is not substantially endocytosed or degraded by cell receptor binding in the subject, thereby retaining anti-viral activity in vivo.

5.2 Periodate Treated and Co-Charged Anionic Polysaccharides

The invention encompasses sulfated polysaccharides that have been manipulated or substituted to reduce endocytosis by cell receptors and to increase the flexibility of the polysaccharide backbone to enable the efficient presentation of anionic charged groups to interact with regions on the targeted viruses.

One manipulation encompassed by the present invention is the treatment of sulfated polysaccharides with periodate. Periodate-treated anionic polysaccharides have increased flexibility due to periodate oxidation of some or all sugar residues. This treatment allows increased freedom of rotation and conformational flexibility of the polymers and provide flexible joints to facilitate biological interactions. Periodate-treated sulfated polysaccharides of the invention can have any counterion to ensure solubility including, but not limited to sodium, calcium, quaternary ammonium, and potassium.

Materials which may be periodate treated and used within the methods and compositions described herein also include the polysaccharides of Table 1 above.

Other variations include the substitution of non-sulfate groups, such as carboxymethyl groups and sulfonate groups. By lowering the degree of substitution of charge on the polysaccharide with either sulfonate or carboxymethyl groups, the ability of the polysaccharide to be endocyctosed by high charge receptors is greatly reduced, therefore increasing its plasma stability. Carboxymethyl dextran sulfate can be prepared using a modification of methods of preparation employed by others (McLaughlin and Hirbst, Can. J. Res. 28B; 731-736, 1950; Brown et al. Arkiv Kemi 22: 189-206 1964). Approximately 20 g of dextran is slurried in a mixture of isopropanol (350 ml) and 3.85M NaOH (40 ml) and is stirred for five minutes at 5° C. in a blender. Sodium chloroacetate (18 g) is added, and the whole mixture is stirred for 60 minutes at 5° C. under a nitrogen atmosphere, the mixture is removed from the blender and stored at 25° C. for three days. The degree of carboxymethyl substitution can be adjusted by varying the time at 25° C. from 1 day to 3 days as well as varying the mole ratio of CICH₂COONa to anhydroglucose from 1 to 4 and keeping the molar ratio of ClCH₂COONa to NaOH to 1 to 1.4. After neutralization the sample is washed with 80% ethanol and dried.

In a preferred embodiment, the invention encompasses a method of treating, managing or preventing a viral infection in a subject (preferably, a human) which comprises administering to a subject in need thereof an effective amount of a co-charged anionic polysaccharide which has a percent of sulfation which enables maximal interaction with the virus and which is not substantially endocytosed or degraded by cell receptor binding in the mammal thereby retaining antiviral in vivo. In a particular embodiment, co-charged anionic polysaccharide is co-charged with carboxymethyl groups, sulfonate groups, sulfate groups or mixtures thereof; more preferably the co-charged anionic polysaccharide is co-charged with carboxymethyl groups. In a specific embodiment, the co-charged anionic polysaccharide is carboxymethyl dextran sulfate or carboxymethyl cellulose.

5.3 Methods of Treatment, Prevention and Management Of Microbial Infections

The invention provides the use of the sulfated polysaccharide and/or substituted polysaccharides (e.g. co-charged anionic polysaccharide) to prevent, treat and/or manage a viral infection. Viral infections which can be treated, prevented or managed by the methods of the present invention include, but are not limited to infections by DNA and RNA viruses. The DNA and RNA viruses within the scope of the invention include, but are not limited to double-stranded DNA viruses, single-stranded DNA viruses, DNA reverse transcripting viruses, RNA reverse transcripting viruses, double-stranded RNA viruses, negative-sense single stranded RNA viruses, positive-sense single-stranded RNA viruses, and ambisense RNA viruses. In one specific embodiment, the methods and compositions can be used to treat, prevent or manage infection of non-enveloped viruses, including but not limited to, picomaviruses, caliciviruses, astroviruses, reoviruses, bimaviruses, circoviruses, parvoviruses, papovaviruses, and adenoviruses.

In preferred specific embodiment, the methods and compositions can be used to treat, prevent or manage infection of enveloped viruses, including but not limited to, togaviruses, flaviviruses, rhabdoviruses, filoviruses, paramyxoviruses, orthomyxoviruses, bunyaviruses, arenaviruses, retroviruses, hepadnaviruses, herpesviruses, poxviruses, iridoviruses, and arteriviruses.

In a specific embodiment, the methods and compositions of the invention are not used to treat, prevent or manage infection of retroviruses, herpesviruses, papilomaviruses paramyxoviruses, orthomyxoviruses, tropical viruses, cytomegaloviruses, or unconventional viruses.

In a more specific embodiment, the methods and compositions of the invention are not used to treat, prevent or manage infection of HIV, Herpesvirus, hepatitis virus, HTLV, BLV, FLV, FeLV, FIV, visna-maedi virus, goat arthritus virus, human spumavirus, HPV-11, HPV-40, CMV, HCMV, RSV, influenza, coxsackie virus B, echo 6, junin virus, tacaribe virus, classical swine fever virus, SCID, African swine fever virus, or Venezuelan equine encephalomyelitis virus.

Specific enveloped double-stranded DNA virus infections which can be treated, prevented or managed by the methods of the present invention include, but are not limited to, Herpesvirus B virus (Cercopithecus herpesvirus 1), Cowpox virus, Epstein-Barr virus (human herpesvirus 4), Hepatitis B virus, Herpes simplex viruses 1 and 2 (HSV-1 and -2), Human cytomegalovirus (human herpesvirus 5), Human herpesviruses 6A, 6B and 7, Molluscum contagiosum virus, Monkeypox virus, Pseudocowpox virus, Tanapox virus, Vaccinia virus, Varicella-zoster virus, Variola virus (smallpox virus), African swine fever virus, Bovine mamillities virus, Bovine papular stomatitis virus, Chelonoid herpesvirus 1, Cowpox virus, Ectromelia virus (mousepox virus), Equine abortion virus (EHV1), Equine coital exanthema virus (EHV3), Equine rhinopneumonitis virus (EHV4), Fibroma viruses (of rabbits, hares and squirrels), Frog viruses 1-3,5-24, L2, L4, and L5, Fowlpox virus, Goldfish viruses 1-2, Infectious bovine rhinotracheitis virus, Infectiuos bovine rhinotracheitis virus, Infectious laryngotracheitis virus (fowl), Lymphocystis disease virus (fish), Marek's disease virus (fowl), Movar herpesvirus, Myxoma virus, Orf virus (contagious pustular dermatitis virus), Pseudocowpox virus (milker's nodule virus), Pseudorabies virus, Sheeppox virus, Swinepox virus, Yabapox virus, and Woodchuck hepatiitis virus.

Specific non-enveloped double-stranded DNA virus infections which can be treated, prevented or managed by the methods of the present invention include, but are not limited to Adenovirus 1-49, Simian adenoviruses 1-27, Bovine adenoviruses 1-9, Porcine adenoviruses 1-4, Ovine adenoviruses 1-6, Equine adenoviruses 1-2, Murine adenoviruses 1-2, BK virus, JC virus, K virus (rabbits), Rabbit kidney vacuolating virus, Papillomaviruses 1-60, Simian virus 12 (SV 12), Simian virus 40 (SV 40), Bovine papillomaviruses 1, 2, and 4, Canine oral papillomavirus, Canine adenovirus 2, equine papillomavirus, ovine papillomavirus, Equine adenoviruses, Fetal rhesus kidney virus, Infectious canine hepatitis virus, Mouse polyoma virus, African green monkey B-lymphotropic polyoma virus, and Shope papillomavirus.

Specific non-enveloped single-stranded DNA virus infections which can be treated, prevented or managed by the methods of the present invention include, but are not limited to Parvovirus B-19, RA-1 virus, Aleutian mink disease virus, Canine parvovirus, Mink enteritis virus, Minute virus of mice, Chicken anemia virus, Psittacine beak and feather disease virus, and Porcine circovirus.

Specific non-enveloped single-stranded positive sense RNA virus infections which can be treated, prevented or managed by the methods of the present invention include, but are not limited to Coxsackieviruses A 1-21 and A24, Coxsackieviruses B1-6, Echoviruses 1-7, 9, 11-27 and 29-34, Enteroviruses 68-71, Hepatitis A virus, Hepatitis E virus, Norwalk and similar viruses (such as Southampton, Snow Mountain, Hawaii, and Taunton viruses), Polioviruses 1-3, Rhinoviruses 1-113, 1A, and 1B, Bovine enteroviruses 1-7, Encephalomyocarditis virus, Feline calicivirus, Foot-and-mouth disease viruses, Mouse poliomyelitis virus (Theiler's virus), Murine encephalomyelitis virus, Porcine enteroviruses 1-8, Bovine enteroviruses 1-7, Simian enteroviruses 1-18, Rabbit hemorrhagic disease virus, Swine vesicular disease virus, Vesicular exanthema viruses 1-12 (swine), Chimpanzee calicivirus (Pan-1), San Miguel sea lion viruses 1-8, European brown hare disease virus, Feline calicivirus, Canine calicivirus, Bovine enteric calicivirus, Porcine enteric calicivirus, Mink calicivirus, Reptile calicivirus, Walrus calicivirus, Fowl calicivirus, Human astroviruses 1-5, Bovine astroviruses 1-2, Ovine astrovirus, Porcine astrovirus, Canine astrovirus, and Duck astrovirus.

Specific enveloped single-stranded positive sense RNA virus infections which can be treated, prevented or managed by the methods of the present invention include, but are not limited to Barmah Forest virus, Central European encephalitis virus, Chikungunya virus, Dengue viruses 1-4, Eastern equine encephalitis virus, Hepatitis C virus, Human immunodeficiency viruses 1 and 2, Human T-lymphotropic viruses 1 and 2, Igbo Ora virus, Japanese encephalitis virus, Kyasanurforest virus, Mayaro virus, Murray Valley encephalitis virus, O'nyong-nyong virus, Omsk hemorrhagic fever virus, Rocio virus, Ross River virus, Rubella virus, Russian spring-summer encephalitis virus, Semliki Forest virus, Sindbis virus (and variants Ockelbo and Babanki viruses), St. Louis encephalitis virus, Venezuelan equine encephalitis virus, West Nile virus, Western equine encephalitis virus, Yellow fever virus, Avian reticuloendotheliosis virus, Avian sarcoma and leukosis viruses, Border disease virus (sheep), Bovine immunodeficiency virus, Bovine leukemia virus, Bovine diarrhea virus, Caprine arthritis-encephalitis virus, Classical swine fever virus, Eastern equine encephalitis virus, Equine infectious anemia virus, Feline immunodeficiency virus, Feline leukemia virus, Feline sarcoma virus, Getah virus, Hog cholera virus, Japanese encephalitis virus, Lactic dehydrogenase-elevating virus (mice), Maedi/visna virus (sheep), Mouse hepatitis viruses, Mouse mammary tumor virus, Mucosal disease virus (cattle), Murine leukemia viruses (including Abelson, AKR, Friend, Maloney leukemia viruses, Progressive pneumonia virus of sheep, Rous sarcoma virus, Rauscher murine leukemia virus, Simian Immunodeficiency viruses (including African Green Monkey, Sooty mangabey, Stump-tailed macaque, pig-tailed macaque, Rhesus, Chimpanzee, and Mandrill viruses), Simian Type D retrovirus, Simian T-cell lymphotrophic viruses, Tick-borne encephalitis viruses (including European and far eastern tick-borne encephalitis viruses, Louping ill virus, and Powassan virus), Venezuelan equine encephalitis virus, Wesselsbron virus, and Western equine encephalitis virus, and Woolly monkey sarcoma virus.

Specific enveloped single-stranded negative sense RNA virus infections which can be treated, prevented or managed by the methods of the present invention include, but are not limited to Alagoas virus, Bunyamwera virus, Bwamba virus, California encephalitis virus, Congo-Crimean hemorrhagic fever virus, Chandipura vitus, Duvenhage virus, Guama virus, Guanarito virus, Hantaan virus, Influenza viruses A, B, and C, Isfahan virus, Jamestown Canyon virus, Junin virus (Argentine hemorrhagic fever virus), Lagos bat virus, La Crosse virus, Lassa virus, Lymphocytic choriomeningitis virus (LCM virus), Machupo virus, Maraba virus, Marburg virus, Measles virus, Mumps virus, Mokola virus, Muerto Canyon virus, Oriboca virus, Oropouche virus, Parainfluenza viruses I (Sendai virus), 2, 3, 4a, and 4b, Pichinde virus, Piry virus, Punto toro virus, Puumala virus, Rabies virus, Respiratory syncytial virus, Rift Valley fever virus, Sandfly fever-Naples virus, Sandflyfever-Sicilian virus, Seoul virus, Sin Nombre virus, Tacaribe virus, Tahyna virus, Tamiami virus, Vesicular stomatitis viruses (including New Jersey and Indiana strains), Akabane virus, Aino virus, Avian paramyxovirus 2 (Yucaipa virus), 3, 4, 5 (Kunitachi virus), 6, 7, 8, and 9, Bovine ephemeral fever virus, Bovine respiratory syncytial virus, Canine distemper virus, Dolphin and Porpoise distemper virus, Ebola virus (including subtypes Zaire, Sudan, and Reston), Equine morbillivirus, Infectious hematopoietic necrosis virus (fish), Influenza viruses of swine, horses, seals, and fowl, Kotonkan virus, Lymphocytic choriomeningitis virus, Marburg virus, Nairobi sheep disease virus, Newcastle disease virus (fowl), Obodhiang virus, Peste-des-petits-ruminants virus (sheep and goats), Pneumonia virus of mice, Pocine rubulavirus (la-Piedad-Michoacan-Mexico virus), Rabies virus, Rift Valleyfever virus, Rinderpest virus, Simian parainfluenza virus 10, and Vesicular stomatitis viruses.

Specific double-stranded RNA virus infections which can be treated, prevented or managed by the methods of the present invention include, but are not limited to Colorado tickfever virus, Reoviruses 1-3, Orungo virus, Kemerovo virus, Rotavirus groups A-F, Eyach virus, Ibaraki virus, Golden shiner virus, chub reovirus, African horsesickness viruses 1-9, Epizootic hemorrhagic disease viruses (deer), Infectious bursal disease virus (fowl), Infectious pancreatic necrosis virus (fish), Human rotaviruses, and Reoviruses 1-3.

In one embodiment, the invention encompasses the treatment, prevention or management of infections by viruses that cause, lead to or are involved in cancer. Further, the invention encompasses the treatment, prevention or management of infections by viral strains that are resistant to or exhibit resistance to conventional antiviral therapy.

In a specific embodiment of the invention, the methods and compositions of the invention are not used to treat, prevent or manage a herpes virus, or more specifically, the methods and compositions of the invention are not used to treat, prevent or manage HSV-1 infection and/or HSV-2 infection. Further, in another embodiment, the methods and compositions of the invention are not used to treat, prevent or manage a retrovirus, or more specifically, the methods and compositions of the invention are not used to treat, prevent or manage HIV-1 infection, HIV-2 infection and/or HTLV infection. Further, in another embodiment, the methods and compositions of the invention are not used to treat, prevent or manage a hepatitis B virus, HCMV, MCMV, VZV, EBV, Measles virus, Punto Toro a, VEE, West Nile Virus, Vaccinia, Cowpox, Adenovirus Type 1, HPIV, Human metapneumoviurs, Haemorrhagic septicaemia virus, Parainfluenza type 3, Pichinde and/or rhinovirus infection.

The sulfated polysaccharides and/or substituted polysaccharides (e.g., a co-charged anionic polysaccharide) of the invention can be used to treat, prevent and/or manage non-viral, microbial infections, including, but not limited to bacterial infections, parasitic infections and fungal infections. Bacterial infections that may be treated, prevented or managed by the methods of as described herein include both gram positive infections and gram negative infections. Specific bacterium that may be treated, prevented or managed by the methods as described herein include, but are not limited to, Chlamydia trachomatis; Helicobacter pylori; Lactobacilli; Escherichia coli; Staphylococcus aureus; Staphylococcus epidermis; Staphylococcus hemolyticus; Saccharomyces cerevisiae; Pseudomonas aeruginosa; Legionella pneumophila; Neisseria gonorrhea; and Neisseria meningitidis.

Fungal infections that may be treated, prevented or managed by the methods of as described herein include but are not limited to, candida sepsis; candida albicans; allergic bronchopulmonary aspergillosis; pulmonary cryptococcus; pulmonary cryptococcus neoformans; invasive pulmonary aspergillosis; candida keratomycosis; cryptococcal meningitis; ascending pyelonephritis due to candida albicans; and candida.

Parasitic infections that may be treated, prevented or managed by the methods of as described herein include but are not limited to, those described in The Handbook of Animal Models of Infection (Zak and Sande eds., Academic Press; 1st edition (1999), including but not limited to, Plasmodium sp. (e.g. Plasmodium Knowlesi and Plasmodium falciparum), Trichomonas vaginalis, hookworm, ringworm, roundworm, whipworm, tapeworm, and pinworm. Specific examples parasitic infections that may be treated, prevented or managed in accordance with the methods of the inventions include, but are not limited to, protozoans (e.g., chilomastix, mesnili, Dientamoeba fragilis, trichomonas, vaginilis, giardea lamblia, cryptobla sp., trypanosma spp., trypanosma cruzi, leishmania spp. and sarcodina), apicomplexa, myxozoa, ciliophora, plathyhelminthes, (e.g. temnociphala sp., gyrodactylus sp., tetraonchus sp., microcotyle sp., octomacroon sp., polystoma sp., polystomoides sp., rajonchocotyle sp., aspidogastec sp., cotylaspis sp., dictyangivm sp., troglotrematidae, and proterometra sp.), and mematodes.

The present invention provides methods for introducing an effective amount of a sulfated polysaccharide or combination of such sulfated polysaccharides into the blood stream, lymphatic system, and/or extracellular spaces of the tissue of a patient in the treatment and/or prevention of microbial infections (e.g., viral infections, bacterial infections or parasitic infections). With respect to viral infections, the method comprises administering to a subject (preferably, a human) at least sulfated polysaccharide that exhibits anti-viral activity in vitro, the sulfated polysaccharide having a sulfation which results in retention of anti-viral activity of the charged polysaccharide in vivo, e.g., sulfation that minimizes uptake by cells that have high charge density cell receptors.

Without being limited by theory, the Applicant believes that the sulfated polysaccharides of the invention have a high affinity for the lymph nodes thus have an increased activity against viruses which populate or gestate in the lymphatic system. Thus, the present invention encompasses a method of administering a sulfated polysaccharide of the invention directly to or targeted for the lymphatic system of a patient.

The magnitude of a prophylactic or therapeutic dose of a sulfated polysaccharide of the invention or a pharmaceutically acceptable salt, solvate, hydrate, or stereoisomer thereof in the acute or chronic management of an infection or condition will vary, however, with the nature and severity of the infection, and the route by which the active ingredient is administered. The dose, and perhaps the dose frequency, will also vary according to the disease or infection to be treated, the age, body weight, and response of the individual patient. Suitable dosing regimens can be readily selected by those skilled in the art with due consideration of such factors.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of a microbial infection (e.g., a viral infection, a fungal infection, a bacterial infection and/or a parasite infection). These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to or non-responsive to therapy such as resistance or non-responsivity to reverse transcriptase inhibitors, protease inhibitors, etc.

5.3.1 Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lie preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are susceptible to infection with the virus to be treated, prevented, or managed (e.g. primary cells, transformed cell lines, patient tissue samples, etc) or growth medium on which the virus to be treated, prevented, or managed can grow (e.g., LB broth/agar, YT broth/agar, blood agar, etc.) are exposed to or otherwise administered a compound of the invention and the effect of the compound upon the ability of the virus to grow is assessed. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a sulfated polysaccharide or a substituted polysaccharide (e.g., a co-charged anionic polysaccharide) of the invention or a pharmaceutically acceptable salt, solvate, hydrate, or stereoisomer thereof in the acute or chronic management of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. In general, the dose per day is in the range of from about 0.001 to 500 mg/kg, preferably about 0.01 to 200 mg/kg, more preferably about 0.005 to 100 mg/kg. For treatment of humans infected by viruses, about 0.1 mg to about 15 g per day is administered in about one to four divisions a day. Additionally, the recommended daily dose ran can be administered in cycles as single agents or in combination with other therapeutic agents. In one embodiment, the daily dose is administered in a single dose or in equally divided doses.

Different prophylactically and therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

5.3.2 Combination Therapy

Specific methods of the invention further comprise the administration of an additional therapy (i.e., a prophylactic agent or therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, β-interferons, alkylating agents, hormones or cytokines.

The sulfated polysaccharides and/or substituted polysaccharides (e.g., a co-charged anionic polysaccharide) of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)),aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The sulfated polysaccharides and/or substituted polysaccharides (e.g., a co-charged anionic polysaccharide) of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinols, thiethylperazine, thioproperazine, tropisetron, and mixtures thereof.

The sulfated polysaccharides and/or substituted polysaccharides (e.g., a co-charged anionic polysaccharide) of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The sulfated polysaccharides and/or substituted polysaccharides (e.g., a co-charged anionic polysaccharide) of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The sulfated polysaccharides and/or substituted polysaccharides (e.g., a co-charged anionic polysaccharide) of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The sulfated polysaccharides and/or substituted polysaccharides (e.g., a co-charged anionic polysaccharide) of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, and ribavirin, as well as foscamet, amantadine, rimantadine, saquinavir, indinavir, amprenavir, lopinavir, ritonavir, the alpha-interferons; adefovir, clevadine, entecavir, pleconaril.

The sulfated polysaccharides and/or substituted polysaccharides (e.g., a co-charged anionic polysaccharide) of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boeringer), IDEC-CE9.1® ([DEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-αreceptor or a fragment thereof, the extracellular domain of an IL-1β receptor or a fragment thereof, and the extracellular domain of an IL-6 receptor or a fragment thereof), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The sulfated polysaccharides and/or substituted polysaccharides (e.g., a co-charged anionic polysaccharide) of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (1N), e.g., IFN-alpha, and IFN-gamma).

The sulfated polysaccharides and/or substituted polysaccharides (e.g., a co-charged anionic polysaccharide) of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The sulfated polysaccharides and/or substituted polysaccharides (e.g., a co-charged anionic polysaccharide) of the invention can be administered or formulated in combination with β-interferons which include, but are not limited to, interferon beta-1a and interferon beta-1b.

The sulfated polysaccharides and/or substituted polysaccharides (e.g., a co-charged anionic polysaccharide) of the invention can be administered or formulated in combination with an absorption enhancer, particularly those which target the lymphatic system, including, but not limited to sodium glycocholate; sodium caprate; N-lauryl-ÿ-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more sulfated polysaccharides of the invention and one or more absorption enhancers.

The sulfated polysaccharides and/or substituted polysaccharides (e.g., a co-charged anionic polysaccharide) of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other prophylactic or therapeutic agents can act additively or, more preferably, synergistically. In a preferred embodiment, a composition comprising a compound of the invention is administered concurrently with the administration of another prophylactic or therapeutic agent, which can be part of the same composition or in a different composition from that comprising the compounds of the invention. In another embodiment, a compound of the invention is administered prior to or subsequent to administration of another prophylactic or therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more sulfated polysaccharides and/or substituted polysaccharides (e.g., a co-charged anionic polysaccharide) of the invention without an additional therapeutic agent. In a specific embodiment, the methods of the invention comprise the administration of one or more sulfated polysaccharides of the invention without a fibroblast growth inhibitor.

5.4 Pharmaceutical Compositions and Dosage Forms

Pharmaceutical compositions and single unit dosage forms comprising a sulfated polysaccharide or substituted polysaccharides (e.g., a co-charged anionic polysaccharide) of the invention, or a pharmaceutically acceptable salt, hydrate or stereoisomer thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients. Sterile dosage forms are also contemplated.

In an alternative embodiment, the pharmaceutical composition encompassed by this embodiment include one or more sulfated polysaccharide of the invention, or a pharmaceutically acceptable salt, hydrate or stereoisomer thereof, and at least one additional therapeutic agent. In an another embodiment, the pharmaceutical composition encompassed by this embodiment include one or more substituted polysaccharides (e.g., a co-charged anionic polysaccharide) of the invention, or a pharmaceutically acceptable salt, hydrate or stereoisomer thereof, and at least one additional therapeutic agent. In yet another embodiment, the pharmaceutical composition encompassed by this embodiment include one or more sulfated polysaccharides and one or more substituted polysaccharides (e.g., a co-charged anionic polysaccharide) of the invention, or a pharmaceutically acceptable salt, hydrate or stereoisomer thereof, and at least one additional therapeutic agent. Examples of additional therapeutic agents include, but are not limited to, those listed above in section 5.3.2.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise sulfated polysaccharides of the invention, or a pharmaceutically acceptable salt, hydrate, or stereoisomers thereof comprise 0.1 mg to 1500 mg per unit to provide doses of about 0.01 to 200 mg/kg per day.

5.4.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103Tm and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.4.2 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.4.4 Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.5 Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). In a preferred embodiment of the invention, the sulfated polysaccharides of the invention have a molecular weight greater than about 500,000 when administered topically.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

5.4.6 Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The sulfated polysaccharides of the invention may also be administered directly to the lung by inhalation. For administration by inhalation, a sulfated polysaccharide can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a sulfated polysaccharide directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a sulfated polysaccharide to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a sulfated polysaccharide to the lung is a liquid spray device supplied, for example, by Aradigm Corporation. Liquid spray systems use extremely small nozzle holes to aerosolize liquid drug formulations that can then be directly inhaled into the lung.

In a preferred embodiment, a nebulizer device is used to deliver sulfated polysaccharides to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In a particularly preferred embodiment, an electrohydrodynamic (“EHD”) aerosol device is used to deliver sulfated polysaccharides to the lung. EHD aerosol devices use electrical energy to aerosolize liquid drug solutions or suspensions (see, e.g., Noakes et al., U.S. Pat. No. 4,765,539; Coffee, U.S. Pat. No., 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the sulfated polysaccharides formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of sulfated polysaccharides will be known to the skilled artisan and are within the scope of the invention.

Liquid drug formulations suitable for use with nebulizers and liquid spray devices and EHD aerosol devices will typically include a sulfated polysaccharide with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of sulfated polysaccharide. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. Nos. 5,112,598; Biesalski, 5,556,611, which are herein incorporated by reference) A sulfated polysaccharides can also be formulated in rectal or vaginal compositions such as suppositories or retention enemas, e.g., containing conventional suppository bases such as cocoa butter or other glycerides.

In addition to the formulations described previously, a sulfated polysaccharide can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver sulfated polysaccharides. Certain organic solvents such as dimethylsulfoxide can also be employed, although usually at the cost of greater toxicity. A sulfated polysaccharide can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989,25,351; Howard et al., 1989, J. Neurosurg. 71, 105). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.7 Condoms and Prophylactic Devices

The sulfated polysaccharides and/or substituted polysaccharides (e.g., a co-charged anionic polysaccharide) of the invention can be used as a coating for a condom (including female condom), diaphragm, cervical cap, dental dam, intrauterin device (IUD) or other prophylactic device. Similarly, the sulfated polysaccharide and/or substituted polysaccharide (e.g., a co-charged anionic polysaccharide) can be used as a coating for surgical instruments and protective devices including, but not limited to rubber gloves, surgical masks, CPR aids, tongue depressors, bandages, sponges, napkins, dental devices and thermometer probe covers. When a sulfated polysaccharide of the invention is used as a coating as described herein, it is preferred to have a molecular weight higher than 500,000. In a preferred embodiment, a sulfated polysaccharide of the invention is combined with a talcum lubricant powder used to line surgical gloves. The methods of using the sulfated polysaccharides of the invention as a coating will be well known by the skilled artisan. Similar methods can be found in U.S. Pat. No. 4,869,270 which is incorporated herein by reference.

5.4.8 Disinfectants and Detergents

The sulfated polysaccharides and/or substituted polysaccharides (e.g., a co-charged anionic polysaccharide) of the invention can be used to disinfect inanimate objects in hospitals, laboratories, lavatories, auditoriums, stadiums, convention centers, restaurants, fitness centers, subway terminals, bus terminals, airports, post offices, offices, sewage treatment facilities, sewers, water treatment facilities, pumping stations, automobiles, airplanes, trains, homes, lockers, and furniture to prevent the spread of viruses or disease.

Disinfectant compositions comprise one or more sulfated polysaccharides or substituted polysaccharides (e.g., a co-charged anionic polysaccharide) of the invention in the form of powders, pastes, concentrates, solutions, sprays, soaps, foams, gels, lotions, creams, handwashes, mouthwashes, pretreated towels, pretreated towelettes, pretreated cotton swabs, or pretreated pads.

In a specific embodiment, the sulfated polysaccharides and/or substituted polysaccharides (e.g., a co-charged anionic polysaccharide) of the invention can be used to disinfect biological fluid including, but not limited to blood, plasma, ova, sperm, or semen. The sulfated polysaccharides and/or substituted polysaccharides (e.g., a co-charged anionic polysaccharide) of the invention can be added directly to the biological fluid or coupled to a solid support, including, but not limited to plastic beads, glass beads, or filters which is placed in contact with the biological fluid.

5.4.9 Nutritional Products and Dietary Supplements

The sulfated polysaccharides and/or substituted polysaccharides (e.g., a co-charged anionic polysaccharide) may be incorporated into nutritional products including, but not limited to food compositions, over the counter, and dietary supplements. The sulfated polysaccharides and/or substituted polysaccharides (e.g., a co-charged anionic polysaccharide) may be added to various foods so as to be consumed simultaneously. As a food additive, the sulfated polysaccharides and/or substituted polysaccharides (e.g., a co-charged anionic polysaccharide) of the invention may be used in the same manner as conventional food additives, and thus, only needs to be mixed with other components to enhance the taste. Taste enhancement includes, but is not limited to, imparting to food a refreshingness, vitality, cleanness, fineness, or bracingness to the inherent taste of the food.

It will be recognized that dietary supplements may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above. The tablets may be coated by methods well-known in the art. In a preferred embodiment, the dietary supplement may take the form of a capsule or powder to be dissolved in a liquid for oral consumption.

The amount of sulfated polysaccharides and/or substituted polysaccharides (e.g., a co-charged anionic polysaccharide) in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises sulfated polysaccharides in an amount of about 1% to about 10% by weight of the food composition.

Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

5.5 Assays and Animal Models

The sulfated polysaccharides and/or substituted polysaccharides (e.g., a co-charged anionic polysaccharide), compositions and dosage forms of the invention can be tested in vitro or in vivo by a variety of methods known in the are to test antiviral activity. See, for example, the methods discussed below and used throughout the examples.

A number of assays may be employed in accordance with the present invention in order to determine the degree of anti-viral activity of a sulfated polysaccharide of the invention such as cell culture, animal models, and administration to human subjects. The assays described herein may be used to assay viral growth over time to determine the growth characteristics of a virus in the presence of a sulfated polysaccharide of the invention.

In one embodiment, a virus and a sulfated polysaccharide a sulfated polysaccharide or substituted polysaccharide (e.g., a co-charged anionic polysaccharide) of the invention are added to a permissive cell line (e.g. primary cells, transformed cell lines, patient tissue samples, etc) or growth medium (e.g., LB broth/agar, YT broth/agar, blood agar, etc). The growth/infection of the virus can be compared the growth/infection of the virus in the absence of the sulfated polysaccharide or substituted polysaccharide of the invention. Anti-virus activity of the sulfated or subsituted polysaccharide of the invention is demonstrated by a decrease in virus growth/infection in the presence of the sulfated or substituted polysaccharide of the invention. Any method known in the art can be used to determine the growth/infection including, but not limited to, immunofluorescent staining, immunoblot or detection of a virus-specific nucleic acid (e.g., by in situ hybridization, or after cell lysis by Southern blot or RT-PCR analysis), visual/microscopic inspection for cytopathic effect of growth/infection (e.g., cell rounding, cell detachment, cell lysis, formation of multinucleated syncytia), virus titer (e.g., plaque forming units, colony forming units, etc.), number of plaques/colonies. In a specific embodiment, the virus and the sulfated or substituted polysaccharide of the invention are added to the cells or growth medium at the same time. In another specific embodiment, the virus is added to the cells or growth medium before the sulfated or substituted polysaccharide of the invention. In another specific embodiment, the sulfated or substituted polysaccharide of the invention is added to the cells or growth medium before the virus.

In another embodiment, a virus and a sulfated polysaccharide or substituted polysaccharide (e.g., a co-charged anionic polysaccharide) of the invention are administered to animal subjects susceptible to infection with the virus. The incidence, severity, length, virus load, mortality rate of infection, etc. can be compared to the incidence, severity, length, virus load, mortality rate of infection, etc. observed when subjects are administered the virus alone (in the absence of a sulfated or substituted polysaccharide of the invention). Anti-virus activity of the sulfated or substituted polysaccharide or substituted polysaccharide (e.g., a co-charged anionic polysaccharide) of the invention is demonstrated by a decrease in incidence, severity, length, virus load, mortality rate of infection, etc. in the presence of the sulfated or substituted polysaccharide of the invention. In a specific embodiment, the virus and the sulfated polysaccharide or substituted polysaccharide (e.g., a co-charged anionic polysaccharide) of the invention are administered to the animal subject at the same time. In another specific embodiment, the virus is administered to the animal subject before the sulfated polysaccharide or substituted polysaccharide (e.g., a co-charged anionic polysaccharide) of the invention. In another specific embodiment, the sulfated polysaccharide or substituted polysaccharide (e.g., a co-charged anionic polysaccharide) of the invention is administered to the animal subject before the virus.

In another embodiment, the growth rate of the virus can be tested by sampling cell culture medium or biological fluids/clinical samples (e.g., nasal aspirate, throat swab, sputum, broncho-alveolar lavage, urine, saliva, blood, or serum) from human or animal subjects at multiple time points post-infection either in the presence or absence of a sulfated or substituted polysaccharide of the invention and measuring levels of virus. In specific embodiments, the growth rate of a virus is assayed by assessing the presence of virus in a sample after growth in cell culture, growth on a permissible growth medium, or growth in subject using any method well-known in the art, for example, but not limited to, immunoassay (e.g., ELISA; for discussion regarding ELISAs see, e.g., Ausubel et al., eds, 1994, Current Protocols in Molecular Biology, Vol. 1, John Wiley & Sons, Inc., New York at 11.2.1), immunofluorescent staining, or immunoblot analysis using an antibody which immunospecifically recognizes the virus to be assayed or detection of a virus-specific nucleic acid (e.g., by Southern blot or RT-PCR analysis, etc.).

In other specific embodiments, the growth rate of a virus is assayed after growth in a subject. Standard models of in vivo antiviral activity include, but are not limited to, a primo-infection cynomolgus monkey model (Le Grand et al., Symp. Nonhum Primate Models AIDS. 1993 Sep 19-22, 11); and those described in The Handbook of Animal Models of Infection (Zak and Sande eds., Academic Press; 1st edition (1999), including but not limited to a Cytomegalovirus infections guinea pig model; a cytomegalovirus infection rat CMV model; a human cytomegalovirus infection of the SCID-hu (thy/liv) mouse model; an ocular cytomegalovirus infections in SCID-hu mice model; a simian varicella model; a varicella zoster infection of t-cells and skin in the SCID-hu mouse mode; a mouse model of influenza virus infection; a ferret model of influenza virus infection.; a cotton rat model of respiratory syncytial virus; a transgenic mouse models for HBV infections; a duck model for hepatitis B infection; a woodchuck model of hepatitis B virus infection; adult mouse models for rotavirus; a macaques model of SIV infection; a SCID-hu thy-liv mouse models for HIV infection; and a chimpanzee model of HIV-1 infection.

In a specific embodiment, viral titers can be determined by obtaining cell culture medium or biological fluids/clinical samples from infected cells or an infected subject, preparing a serial dilution of the sample and infecting a monolayer of cells that are susceptible to infection with the virus (e.g. primary cells, transformed cell lines, patient tissue samples, etc) at a dilution of the virus that allows for the emergence of single plaques. The plaques can then be counted and the viral titer expressed as plaque forming units per milliliter of sample.

In one specific embodiment, the growth rate of a virus in a subject can be estimated by the titer of antibodies against the virus in the subject. Antibody serum titer can be determined by any method well-known in the art, for example, but not limited to, the amount of antibody or antibody fragment in serum samples can be quantitated by, e.g., ELISA. Additionally, in vivo activity of a sulfated or substituted polysaccharide can be determined by directly administering the sulfated or substituted polysaccharide to a test animal, collecting biological fluids (e.g., nasal aspirate, throat swab, sputum, broncho-alveolar lavage, urine, saliva, blood, or serum) and testing the fluid for anti-virus activity.

In embodiments where samples to be assayed for virus levels are biological fluids/clinical samples (e.g., nasal aspirate, throat swab, sputum, broncho-alveolar lavage, urine, saliva, blood, or serum), the samples may or may not contain in tact cells. Samples from subjects containing intact cells can be directly processed, whereas isolates without intact cells may or may not be first cultured on a permissive cell line (e.g. primary cells, transformed cell lines, patient tissue samples, etc) or growth medium (e.g., LB broth/agar, YT broth/agar, blood agar, etc.). Cell suspensions can be cleared by centrifugation at, e.g., 300×g for 5 minutes at room temperature, followed by a PBS, pH 7.4 (Ca⁺⁺ and Mg⁺⁺ free) wash under the same conditions. Cell pellets can be resuspended in a small volume of PBS for analysis. Primary clinical isolates containing intact cells can be mixed with PBS and centrifuged at 300×g for 5 minutes at room temperature. Mucus is removed from the interface with a sterile pipette tip and cell pellets can be washed once more with PBS under the same conditions. Pellets can then be resuspended in a small volume of PBS for analysis.

In another embodiment, a sulfated or substituted polysaccharide of the invention is administered to a human subject infected with a virus. The incidence, severity, length, viral load, mortality rate of infection, etc. can be compared to the incidence, severity, length, viral load, mortality rate of infection, etc. observed in human subjects infected with a virus in the absence of a sulfated or substituted polysaccharide of the invention or in the presence of a placebo. Anti-viral activity of the sulfated or substituted polysaccharide of the invention is demonstrated by a decrease in incidence, severity, length, viral load, mortality rate of infection, etc. in the presence of the sulfated or substituted polysaccharide of the invention. Any method known in the art can be used to determine anti-viral activity in a subject such as those described previously.

The assays described above can be modified by one of skill in the art where the subject is infected by a microbe, including, but not limited to a parasite, fungus or bacterium. In another embodiment, the microbe is a parasite. Standard models of in vivo antiparasitic activity include, but are not limited to, those described in The Handbook of Animal Models of Infection (Zak and Sande eds., Academic Press; 1st edition (1999), including but not limited to, an intravaginal mouse model of Trichomonas vaginalis infection.

Standard models of in vivo antifungal activity include, but are not limited to, those described in The Handbook of Animal Models of Infection (Zak and Sande eds., Academic Press; 1st edition (1999), including but not limited to, a Rodent model of candida sepsis; a generalized candida albicans infection model in the rat; a oropharyngeal and gastrointestinal candid a infection in mice model; a paw oedema model of localized candidiasis; a murine model of allergic bronchopulmonary aspergillosis; a pulmonary cryptococcus infection in mice model; a pulmonary cryptococcus neoformans infection in rats model; a rat model of invasive pulmonary aspergillosis; a rabbit model of candida keratomycosis; a rabbit model of cryptococcal meningitis; a rat models of ascending pyelonephritis due to candida albicans; a rat model of candida vaginal infection; and a murine model of candida vaginal infections.

Standard models of in vivo antibacterial activity include, but are not limited to, those described in The Handbook of Animal Models of Infection (Zak and Sande eds., Academic Press; 1st edition (1999)), including but not limited to, a mouse peritonitis/sepsis model; a murine thigh infection model; a mouse subcutaneous cotton thread model; a mouse peritonitis model; a murine models of peritonitis involving a foreign body; a rat polymicrobial peritonitis infection model; a mouse model of campylobacter jejuni infection; a suckling mouse model of enterotoxigenis escherichia coli infection; a rabbit model of shigellosis; the RITARD rabbit model of intestinal vibrio cholerne infections; a mouse model of helicobacter pylon infection; a ferret model of helicobacter; a hamster model of syphilis; a guinea pig model of acquired and congenital syphilis; a guinea pig model of legionnaires disease; a murine model of tuberculosis; a beige mouse model of disseminated mycobacterium avuim complex infection; an armadillo leprosy model; a mouse model of leprosy; a hamster model of lyme arthritis; a rabbit model of bacterial conjunctivitis; a murine model of bacterial keratitis; the rabbit intrastomal injection model of bacterial keratitis; a gerbil model of acute otitis media; a ginuea pig model of bacterial otitis externa; a chinchilla model of otitis media; a guinea pig model of acute otitis media; a rat model of bacterial epididymitis; a mouse model of mycoplasma genital infections; a mouse model of ascending urinary tract infection; a mouse model of ascending UTI involving short and long-term indwelling catheters; a rat model of subclinical pyelonephritis; a rat model of chronic cystitis; a mouse pneumococcal pneumonia model; a hamster model of mycoplasma pulmonary infections; a rat model of bacterial osteomyelitis of the tibia; a rat model of hematogenous osteomyelitis; a rabbit model of bacterial osteomyelitis of the tibia; a rat model of arthroplasty; a rabbit model of arthroplasty; a mouse model of streptococcal fasciitis; a rabbit model of bacterial endocarditis; an adult rat model of meningitis; and a rabbit model of bacterial meningitis.

Additionally, in vivo activity of a sulfated or substituted polysaccharide can be determined by directly administering the sulfated or substituted polysaccharide to an animal or human subject, collecting biological fluids/clinical samples (e.g., nasal aspirate, throat swab, sputum, broncho-alveolar lavage, urine, saliva, blood, or serum) and testing the biological fluids/clinical samples for anti-viral activity (e.g., by addition to cells in culture in the presence of the virus).

In general, in vivo stability can be determined by a variety of models known to the skilled artisan. In particular, in vivo stability can be determined by a kidney perfusion assay. For either type of analysis, the test sulfated or substituted polysaccharide may be labeled, for example with tritium. A kidney perfusion technique is described in detail in Tay et al. (Am. J. Physiol., (1991), 260: F549-F554). Briefly, rat kidneys, e.g., from male Sprague-Dawley rats, are perfused with 5% bovine serum albumin (BSA) in modified Krebs Henseleit buffer containing amino acids and continually gassed with 95% O₂-5% CO₂. Samples that have been perfused may be subjected to ion-exchange chromatography using, for example, a 19×1/cm² column of sepharose Q. Samples are applied to the column in 6 M urea, 0.05 M Tris, 0.005% (w/v) Chaps, pH 7.0, and eluted with a linear gradient of 0.15-2.5 M NaCl in the same buffer at a flow rate of 0.5 ml/minute. Recoveries using this technique are very good.

### 6. WORKING EXAMPLES

The following examples are for the purpose of illustration only and are not intended as limiting the scope of the invention.

### 6.1 Example 1

### Synthesis of a Sulfated Dextran Having a Sulfation of 9.5%

Dextran T20 (average molecular weight 20,000) was dried in vacuo at 60° C. overnight. The dried compound (100 g) was dissolved in 640 ml formamide (FA). Chlorosulfonic acid (CSA) 80 ml was added to FA 200 ml at a maximum of 45° C. in a 3-necked flask, then cooled in ice-water. The amount of CSA determines the ultimate sulfation of the sulfated dextran (180 ml CSA to 200 ml FA yields approximately 17% sulfur). The CSA/FA mix was slowly added (over two hours) to the dextran at a temperature of 40° C. After all of the CSA/FA was added, the mixture was stirred for 15 minutes at a temperature of 45° C. The mixture was cooled to 25° C. and 28% NaOH was added slowly to give a pH 7.5-8.5 with a maximum temperature of 50° C. For the first precipitation, 3 L of ethanol were added with stirring. Stirring was stopped and the mixture was allowed to stand. The supernatant was decanted and the precipitate was redissolved in 1.5 L of water. For the second precipitation 1.5 L ethanol were added with stifling and then the mixture was allowed to stand for two hours. The supernatant was decanted and the precipitate was redissolved in 900 ml of water, to which 17 g NaCl was added. For the third precipitation 800 ml ethanol were added with stirring and the mixture was allowed to stand for two hours. The optical rotation-maximum was measured. The supernatant was decanted and the precipitate was redissolved in 500 ml water. 2.8 g Na₂HPO₄ and 2.6 g NaH₂PO₄ were added. For the final precipitation 5 L ethanol were added and the precipitate was filtered on a glass filter and dried in vacuo at 50° C.

### 6.2 Example 2

### Periodate Oxidation

Following the modified method of Smith degradation used by Sandy J D, Biochem J., 177: 569-574, 1979; chrondroitin sulfate (240 mg) was dissolved in 0.25M NaClO₄ (47 ml) at room temperature. 5 ml of 0.5 M NalO₄ was added and KOH was used to adjust the mixture to pH 5. The reaction was allowed to proceed in the dark for 72 hours. The mixture was then dialysed in visking tubing to remove the periodate.

### 6.3 Example 3

### Introduction of Anionic Sulfur Groups to Carboxymethyl Dextran

Sulfated Form of Carboxymethyl Dextran (Average MW 20,000) with a Sulfur Content of 9.5%.

Carboxymethyl dextran (CMD) is dried in vacuo at 60° C. overnight. CMD (100 g) is dissolved in 640 ml formamide (FA). Chlorosulfonic acid (CSA) 80 ml is added to FA 200 ml at maximum of 45° C. in a 3-necked flask then cooled in ice-water. The amount of CSA will determine the ultimate sulfur content of CMD (180 ml OSA to 200 ml FA yields approx 17% sulfur). The CSA/FA mix is added slowly (over 2 hours) to CMD at a temperature of 40° C. After all is added the mixture is stirred for 15 minutes at a temperature of 45° C. The mixture is cooled to 25° C. and 28% NaOH is added slowly to give a pH 7.5-8.5 with a maximum temperature of 50° C. For the first precipitation, 3 L of ethanol is added with stirring. Supernatant is decanted and then residue is redissolved in 1.5 L of water. For the second precipitation 1.5 L ethanol is added with stirring and then allowed to stand for 2 hours. Supernatant is decanted and residue is redissolved in 900 ml of water and then added to 17 g NaCl. For the third precipitation 800 ml ethanol is add with stirring and allowed to stand for 2 hours. The optical rotation maximum should be 0.3. Supernatant is decanted and the residue is redissolved in 500 m water. Add 2.8 g Na₂HPO₄ and 2.6 g NaH₂PO₄. For the final precipitation 5 L ethanol is added and filtered on a glass filter and is dried in vacuo at 50° C.

Sulfonated Form of Carboxymethyl Dextran (Average Molecular Weight 20,000).

Step 1. Dissolve 5 g dextran in water. Add 100 mg borohydride stir at room temp. for 30 min.

Step 2. Add sodium hydroxide pellets (10 g) and stir until dissolved and then sulfonate (12 g).

Step 3. Heat at 70° C. for 7 h. After 3 hours add a further 3 g of sulphonate. Continue heating for 4 hours.

Step 4. Neutralise with 5M HCl to pH 7.5 (Total volume(T)=75 ml) and gradually add 200 ml ethanol with good stirring. Stop stirrer and stand 1 hour.

Step 5. Decant supernatant; redissolve in water (T=60 ml) and add 150 ml ethanol with good stirring. Stand 1 hour.

Step 6. Repeat as Step 5.

Step 7. Decant off the supernatant-redissolve the residue in 60 ml water and ppte in 600 ml ethanol. Some concentrated sodium chloride solution may be added to the mixture to aid precipitation.

Step 8. Filter and dry in vacuo. Yield approx. 6 g.

### 6.4 Example 4

### In Vivo Anti-Viral Activity

The in vivo anti-viral activity of dextran sulfate and variants of sulfated dextrans are assessed in a pharmacokinetic study involving single intravenous doses of 60 mg/kg dextran sulfate (DS) given to three male and three female rats. Rats are Sprague-Dawley, previously cannulated in the vena cava. Blood is drawn at various times after injection and is assessed for anti-HIV activity in an acute infectivity cytoprotection assay system utilizing HIV-1 RF virus with CEN-SS cells using the MTS staining method for cell viability (based on Witvrouw et al., J. Acqur. Immun. Def Syndr., 3:343-347, 1990).

### 6.5 Example 5

### Effect on Pro-Thrombin/Thrombin and Activated Partial Thromboplastin Time

The purpose of this study is to evaluate the effects of dextran sulfate on prothrombin time (PT) and activated partial thromboplastin time (aPTT). All specimens are “spiked” with the test compound prior to submission to a Clinical Pathology Laboratory. The specimens are delivered along with reconstituted human plasma purchased from Sigma. Immediately prior to analysis 600 μl of the Sigma human plasma is added to each specimen.

A Bio-Merieux Coag-A-Mate MTX II Analyzer is used to measure Prothrombin Time (PT) and Activated Partial Thromboplastin Time (APTT). The PT reagent is Simplastin L and the APTT reagent is Platelin L; all reagents are obtained from Bio-Merieux. All specimens are run in duplicate. Coagulation control samples are analyzed immediately prior to testing.

| Parameter | Abbreviation | Units | Method |
|---|---|---|---|
| Prothrombin Time | PT | Seconds | Photo-optical hemostasis |
| | | | analyzer |
| Activated partial | APTT | Seconds | Photo-optical hemostasis |
| Thromboplastin | | analyzer | |
| Time | | | |

Specimen Disposition

The PT measuring time starts at five seconds and stopped at 60 seconds. The aPTT measuring time starts at five seconds and stops at 130 seconds.

Thrombocytopenia and Coagulation

Experiments to determine the effect of injected destran sulfate of various molecular weights on coagulation parameters are undertaken. Rats are given either 5 or 50 mg/kg (i.v.) of each compound on consecutive days for ten days. On day 11, dosages are changed from 5 to 1 mg/kg and 50 to 100 mg/kg and daily consecutive intravenous injections are continued. At days 0, 5, 10 and 15 blood is drawn and assessed for aPTT and platelet counts.

Maximum Tolerated Dose

The multiple toxicity dose (MTD) of dextran sulfate is assessed in a series of experiments where groups of five rats are given 100 or 200 mg/kg doses. Body weights and overall behavioral assessments are determined for five days after injection. Subsequently rats are given a 500 mg/kg injection and observed for a further five days. Finally animals are given a dose of 850 mg/kg.

### 6.6 Example 6

### In Vitro Anti-Viral Assessment of Sulfated Polysaccharides

The studies include an assessment of five test compounds at a high test concentration of 500 μg/ml in human peripheral blood mononuclear cells (PBMCs).

Methods

All test compounds are solubilized in H₂O at 40 mg/ml. Compounds are light protected and assays are performed in a manner which minimized incidental light. Compounds are stored at −20° C. following solvation.

Viruses

The low passage pediatric isolate RoJo is derived in the laboratories of Southern Research Institute. RoJo is a presumed subtype B virus.

PBMC Isolation and Blasting

Peripheral blood monocular cells (PBMCs) is obtained from normal hepatitis and HIV-1 negative donors by ficoll hypaque gradient separation. The mononuclear cells are washed to remove residual separation media, counted, viability is determined and is resuspended in RPMI 1640 medium supplemented with 15% FBS (heat inactivated), 2 mM L-glutamine, 100 U/mL penicillin, 100 μg/mL streptomycin, and 10 μg/mL gentamycin with 2 μg/mL phytohemagluttin (PHA) at 1×10⁶ cells/mL. The cells are cultured for 48 to 72 h at 37° C., 5% CO₂. Following incubation, cells are collected by centrifugation, washed and resuspended in RPMI 1640 supplemented with 15% FBS (heat inactivated), 2 mM L-glutamine, 100 U/mL penicillin, 100 μg/mL streptomycin, and 10 μg/mL gentamycin with 20 U/mL recombinant IL-2 R & D Systems, Minneapolis, Minn.). IL-2 is included in the culture medium to maintain the cell division initiated by the PHA mitogenic stimulation. The cultures are then maintained until use by culture volume change with fresh IL-2 containing medium every three days.

PBMC Assay

Human peripheral blood mononuclear cells from a minimum of two donors, that is blasted with PHA and IL-2, are counted, viability is determined by Trypan Blue dye exclusion and mixed in equal ratios. Pooled donors are used to minimize the variability observed between individual donors which results from quantitative and qualitative differences in HIV infection and overall response to the PHA and IL-2 of primary lymphocyte populations. The cells are resuspended at 1×10⁶ cells/mL in RPMI 1640 without phenol red supplemented with 15% Fetal Bovine Serum (heat inactivated), 2 mM L-glutamine, 100 U/mL penicillin, 100 μg/mL streptomycin, 10 μg/μL gentamycin and IL-2 (20 U/mL, R&D Systems, Minneapolis, Minn.). Fifty microliters of cells are then distributed to the inner 60 wells of a 96 well round bottom microtiter culture plate in a standard format developed by the Infectious Disease Research department of Southern Research Institute. Each plate contains cell control wells (cells only), virus control wells (cells plus virus), and experimental wells (drug plus cells plus virus). Serially diluted compounds are added to the microtiter plate followed by the appropriate pretitered dilution of HIV-1 RoJo. All samples are assayed in triplicate with a replicate plate without virus for the determination of compound toxicity. The final volume per well is 200 μL. The assay is incubated for 6 days in a humidified atmosphere at 37° C., 5% CO₂, after which supernatants are collected, for analysis of RT activity and sister plates are analyzed for cell viability by MTS dye reduction. Wells are also examined microscopically and any abnormalities noted.

MTS Staining for Cell Viability

At assay termination the assay plates are stained with the soluble tetrazolium-based dye MTS (CellTiter96® Reagent Promega) to determine cell viability and quantify compound toxicity. MTS is metabolized by the mitochondria enzymes of metabolically active cells to a soluble formazan product, allowing the rapid quantitative analysis cell viability and compound cytotoxicity. This reagent is a single stable solution that does not require preparation before use. At termination of the assay 20 μL of MTS reagent was added per well and incubated for 4 h at 37° C. Adhesive plate sealers are used in place of the lids, the sealed plate is inverted several times to mix the soluble formazan product and the plate is read spectrophotometrically at 490 nm with a Molecular Devices Vmax plate reader.

Reverse Transcriptase Assay for Culture Supernatants

Reverse transcriptase (RT) activity is measured in cell-free supernatants. Tritiated thymidine triphosphate (NEN) (TTP) is resuspended in distilled H₂O at 5 Ci/mL. Poly rA and oligo dT are prepared as a stock solution which was kept at −20° C. The RT reaction buffer is prepared fresh on a daily basis and consists of 125 μL 1.0 M EGTA, 125 μL dH₂O, 110 μL 10% SDS, 50 μL 1.0 M Tris (pH 7.4), 50 μL 1.0 M DTT, and 40 μL 1.0 M MgCl₂. These three solutions are mixed together in a ratio of two parts TTP, one part poly rA:oligo dT, and one part reaction buffer. Ten microliters of this reaction mixture are placed in a round bottom microtiter plate and 15 μL of virus containing supernatant was added and mixed. The plate is incubated at 37° C. in a water bath with a solid support to prevent submersion of the plate and incubated for 60 minutes. Following reaction, the reaction volume is spotted onto pieces of DE81 paper, washed 5 times for 5 minutes each in a 5% sodium phosphate buffer, two times for one minute each in distilled water, two times for one minute each in 70% ethanol, and then dried. Opti-Fluor 0 is added to each sample and incorporated radioactivity was quantitated utilizing a Wallac 1450 Microbetaplus liquid scintillation counter.

Data Analysis

IC₅₀ (50%, inhibition of virus replication), TC₅₀ (50% reduction in cell viability) and a therapeutic index (T₁, TC₅₀/IC₅₀) are provided.

### 6.7 Example 7

### In Vitro Anti-Viral Assessment of Sulfated Polysaccharides

6.7.1 Viruses

Human immunodeficiency virus type 1 (HIV-1) strains Ba-L, ADA, SIVmac251, HIV-2 (CDC3 10319) and the subtype representative strains (Table 2) are obtained from the NIAID AIDS Research and Reference Reagent Program. The low passage pediatric isolates SLKA, WeJo and TeKi are derived in the laboratories of Southern Research Institute. The multi-drug resistant virus MDR-769 is derived from a highly experienced antiretroviral patient and exhibits the resistance profile and genotype outlined in Table 3.

TABLE 2
Subtype Representative Viruses

| Virus | Subtype (env) |
|---|---|
| RW/92/016 | A |
| 302056 (91US056) | B |
| BR/92/025 | C |
| UG/92/046 | D |
| CMU02 | E |
| BR/93/020 | F |
| Jv1083 | G |
| BCOF-01 | O |

TABLE 3
Phenotype and Genotype of the MDR 769 Virus

| | | Other Changes from | Drug |
|---|---|---|---|
| Gene | Resistance Mutations | Consensus B | Resistance |
| RT | M41L, K65R, D67N | K20R, V21I, V35I, K43Q, | AZT, ddI, |
| | V751, F116Y, Q151M, | A62V, E79D, I167I/V, | 3TC, d4T, |
| | Y181I, L210W, T215Y | G196E, Q197K, E207Q, | PFA, NVP |
| | | D218E | |
| PR | L10I, M36M/V, M46I, | V13I, D60E, I62V, K223Q | IDV, |
| | I54V, L63P, A71V, | | SQV, |
| | V82A, I84V, L90M | | NFV |

Mutations in bold face type in Table 3 represent key resistance mutations in the indicated genes.

PBMC Isolation and Blasting

Peripheral blood monocular cells (PBMCs) are obtained as described in Example 6.

PBMC Assay

PBMC assays are carried out as described in Example 6.

### Monocyte Isolation, Culture and Infection

Peripheral blood monocytes are isolated from normal HIV-1 negative donors by plastic adherence following ficoll hypaque purification of the buffy coat, as described above for PBMCs. In many cases the same donor used to produce the PBMC populations is also used to produce monocyte/macrophages, however unlike PBMC population monocyte/macrophage, donors are never pooled. Following a two hour adherence in RPMI 1640 without phenol red supplemented with 10% human pooled AB serum (heat inactivated), 2 mM L-glutamine, 100 U/mL penicillin, 100 μg/mL streptomycin, 10 μg/mL gentamycin, cultures are washed to remove non-adherent cells. The monocytes are released from the plastic by vigorous pipetting with Ca²⁺ and Mg²⁺ free PBS. Adherent cells are assessed for purity by nonspecific esterase staining (a-napthyl butyrate specific esterase, Sigma Chemical Co.), and/or viability by Trypan Blue dye exclusion, counted and resuspended in RPMI 1640 supplemented with 10% Fetal Bovine Serum (heat inactivated), 2 mM L-glutamine, 100 U/mL penicillin, 100 μg/mL streptomycin, 10 μg/mL gentamycin at 1×10⁶ monocytes per ml. The monocytes (1×10⁵ per 0.2 cm well) are then cultured for six days, allowing maturation of the cells to a macrophagelike phenotype. At day six the cultures are washed three times to remove any non-adherent cells and serially diluted test compounds are added followed by the addition of a pre-titered amount of HIV-1 virus, if microscopic observation of the wells demonstrated a 70% or greater confluence of the monocyte/macrophage monolayer. Cultures are washed a final time by media removal 24 hours post infection, fresh compound added and the cultures continued for an additional six days. The assays are preformed using a standardized microtiter plate format, which uses only the inner 60 wells of a 96 well plate for assay purposes. The outer rows contain media and acts an a evaporation barrier. Each plate contains cell control wells (cells only), virus control wells (cells plus virus), and experimental wells (drug plus cells plus virus). HIV p24 antigen content to assess virus replication was measured at assay termination by a commercially available p24 ELISA assay (Coulter) on cell-free supernatants, and compound cytotoxicity by MTS dye reduction. AZT, HIV-1 reverse nucleoside transcriptase inhibitor and dextransulfate, an attachment inhibitor, are used as positive control compounds and run in parallel with each determination. At termination of the assay culture plates were removed from the incubator and observed microscopically. Any unique findings are noted.

MTS Staining for Cell Viability

MTS staining is carried out as described in Example 6.

Reverse Transcriptase Assay fur Culture Supernatants

Reverse transcriptase (RT) activity is measured in cell-free supernatants as described in Example 6.

P24 Antigen ELISA

ELISA kits are purchased from Coulter Electronics. The assay is performed according to the manufacturer's instructions. Control curves are generated in each assay to accurately quantitate the amount of p24 antigen in each sample. Data are obtained by spectrophotometric analysis at 450 nm using a Molecular Devices Vmax plate reader. Final concentrations are calculated from the optical density values using the Molecular Devices Soft Max software package.

6.8 Biodistribution of a Compound of the Invention

Male Sprague-Dawley rats are obtained from Charles River Laboratories (Raleigh, N.C.; ca. 377-402 g) and are dosed with [³H]Des6 40K by intravenous bolus or oral gavage administration. Distribution of total tritium content in plasma, lymph, and cervical lymph nodes is quantitated in samples collected at 6 or 12 hours following dosing.

Rats are divided into three treatment administration groups. Doses are formulated in phosphate buffered saline vehicle (pH=7.4) so as to deliver them in approximate volumes of 1.8 mL/kg (iv) and 2.1 mL/kg (oral gavage).

Prior to the time of biological sample collection at 6 or 12 hours after dosing, animals are anesthetized with ketamine/xylazine (7:1, ca. 120 mg/kg), and the thoracic lymphatic duct is cannulated as described in Waynforth, H. B. and Flecknell, P. A. (1992). Experimental and Surgical Technique in the Rat, 2nd ed., Academic Press, New York. At the time of sample analysis, blood is collected by cardiac puncture and lymph was collected via the lymphatic duct cannula. Blood is processed for plasma by centrifugation at ca. 1000 g for 10 minutes. Cervical lymph nodes are collected from each animal. Total radioactivity is quantitated in duplicate by liquid scintillation spectrometry for all biological samples collected.

The foregoing has demonstrated the pertinent and important features of the present invention. One of skill in the art will be appreciate that numerous modifications and embodiments may be devised. Therefore, it is intended that the appended claims cover all such modifications and embodiments.

1. A method of treating, preventing or managing a viral infection in a human comprising administering to a human in need thereof an effective amount of a naturally occurring, synthetic, or non-synthetic sulfated polysaccharide having a percent of sulfur above 2% but less than 6% or above 13% but less than 25% with respect to the simple sugar residue.

2. The method of claim 1, wherein said sulfated polysaccharide has a percent of sulfur above 13% but less than 22% with respect to the simple sugar residue.

3. The method of claim 2, wherein the percent of sulfur is greater than 14%.

4. The method of claim 3, wherein the percent of sulfur is greater than 17%.

5. The method of claims 3 or 4, wherein the percent of sulfur is less than 20%

6. The method of claim 1; wherein the viral infection is caused by a DNA virus.

7. The method of claim 1, wherein the viral infection is caused by a RNA virus.

8. The method of claim 1, wherein the viral infection is not caused by HSV-1, HSV-2, HIV-1, HIV-2, HTLV, hepatitis B virus, HCMV, MCMV, VZV, EBV, Measles virus, Punto Toro a, VEE, West Nile Virus, Vaccinia, Cow pox, Adenovirus Type 1, HPIV, Human metapneumoviurs, Haemorrhagic septicaemia virus, Parainfluenza type 3, Pichinde, rhinovirus, BLV, FLV, FeLV, FIV, visna-maedi virus, goat arthritus virus, human spumavirus, HPV-11, HPV-40, CMV, HCMV, RSV, influenza, coxsackie virus B, echo 6, junin virus, tacaribe virus, classical swine fever virus, SCID, African swine fever virus, or Venezuelan equine encephalomyelitis virus.

9. The method of claim 6, wherein the DNA virus is a double-stranded DNA virus or a single-stranded DNA virus.

10. The method of claim 7, wherein RNA virus is a double-stranded RNA virus, negative-sense single stranded RNA virus, positive-sense single-stranded RNA virus, or an ambisense RNA virus.

11. The method of claim 1, wherein the sulfated polysaccharide has a molecular weight from about 5,000 to about 1,000,000.

12. The method of claim 1, wherein the sulfated polysaccharide has a molecular weight greater than 25,000.

13. The method of claim 1, wherein the sulfated polysaccharide has a molecular weight greater than 40,000.

14. The method of claim 1, wherein the sulfated polysaccharide comprises D-glucopyranose residues linked by α-1,6 linkages.

15. The method of claim 1, wherein the sulfated polysaccharide comprises L-glucopyranose residues.

16. The method of claim 1, wherein the sulfated polysaccharide is sulfated dextran.

17. The method of claim 1, wherein the sulfated polysaccharide is conventional dextran sulfate.

18. The method of claim 1, wherein the sulfated polysaccharide is dextrin sulfate or carrageenan.

19. A method of treating, preventing or managing a viral infection in a human which comprises administering to a human in need thereof an effective amount of a co-charged anionic polysaccharide, wherein said co-charged anionic polysaccharide has a percent of sulfur above 2% but less than 6% or above 13% but less than 25% with respect to the simple sugar residue and which enables maximal interaction with the virus and which is not substantially endocytosed or degraded by cell receptor binding in the human.

20. The method of claim 19, wherein the co-charged anionic polysaccharide is co-charged with carboxymethyl groups, sulfonate groups, sulfate groups or combinations thereof.

21. The method of claim 20, wherein the co-charged anionic polysaccharide is co-charged with carboxymethyl groups.

22. The method of claim 21, wherein the co-charged anionic polysaccharide is carboxymethyl dextran sulfate or carboxymethyl cellulose.

23. The method of any one of claims 1 or 19, further comprising the administration of an additional therapy or an absorption enhancer.

24. The method of any one of claims 1 or 19, wherein the effective amount is from about 0.001 to 200 mg/kg per day.

25. The method of claim 24, wherein the effective amount of the polysaccharide is from about 0.005 to 100 mg/kg per day.

26. The method of any one of claims 1 or 19, wherein the effective amount of the sulfated polysaccharide is from about 0.1 mg/kg/day to about 1,500 mg/kg/day.

27. The method of any one of claims 1 or 19, wherein the effective amount of the polysaccharide or is administered parenterally.

28. The method of any one of claims 1 or 19, wherein the effective amount of the polysaccharide is administered orally.

29. The method of any one of claims 1 or 19, wherein the effective amount of the polysaccharide is administered mucosally.

30. The method of any one of claims 1 or 19, wherein the effective amount of the polysaccharide is administered into the lung.

31. The method of any one of claims 1 or 19, wherein the effective amount of the polysaccharide is administered topically.

32. A method of treating, preventing or managing a non-viral, microbial infection in a human comprising administering to a human in need thereof a therapeutically effective amount of a naturally occurring, synthetic, or non-synthetic sulfated polysaccharide having a percent of sulfur above 2% but less than 6% or above 13% but less than 25% with respect to the simple sugar residue.

33. A method of treating, preventing or managing a non-viral, microbial infection in a human which comprises administering to a human in need thereof an effective amount of a co-charged anionic polysaccharide, wherein said co-charged anionic polysaccharide has a percent of sulfur above 2% but less than 6% or above 13% but less than 25% with respect to the simple sugar residue.

34. The method of claim 32 or 33 wherein the microbial infection is a bacterial infection.

35. The method of claim 34 wherein the bacterial infection is a gram positive bacterial infection or a gram negative bacterial infection.

36. The method of claim 32 or 33 wherein microbial infection is a fungal infection.

37. The method of claim 32 or 33 wherein the microbial infection is a parasitic infection.
